SEQUENCE LISTING (30 sequences)

SEQ ID NO: 1 - DNA, 2661 bp, Homo sapiens
acagacatgtgccaccatgcccagctaattttttgtttgtttgtttgtttgtttgtatttttagtagagatggggttttgccatgttggccaggctggtctcgaactcctgacctcgaatgataatgatccgccgcttggcctccaaagtgctaggattacaggtgtgagccactgcgccaggcctgggcactttctttagtagtttgaggagcaacatttttgacagtgtccttctgctcaagattcaagatcccagataaaattaaaccatctagagagatggcttgattggccaaacctggatctcatgaccacttcttgaagtgggtaagtctcataaatgctcagtccttccactatgcaactgagtggggtgggtgggaagcccctcaaaggaaaatccggttgttcttactagaaagaaaaggaaaatggatgtgaggcagtcaaaatcagcagaggtccaccacaccaccaaaatgtggtgattaaatatggagagacagagactaacagaggtatgtgaatattgaagtatgtctggacaatagcccaatgatgagaccaataaaatggttaccaaaatctggttttgagtagtagtgttaaatcagaccatttagtaaccattttttgttgcaaagtttctagcactgcccaaaccctgagtggtatatgaataactcgtccattatgtatctctttccagtcagcataatttatcccccacctatattcttttctgaccactcctacttccttctctttaccaaaatctaaactctaaggctgtttcttcagcaacttctttgtttagattggaagataaattaaacagcatgcgatgttttactgactttcagtatttaacagaggtgatttaatttttttttaaatccaaagtcaaacttctttataagatgaaggagaaaaatgtcttataaaatgcatatgtgaagatgccttctgagtgctttctcatgcagacttgttctagtctttaatgaatcttccttgtagacactgtggagatgaaagatggttctccacttctactcaaagtacaaatcaggccggcattttgaaaaagagacaggtttattcatagctgcagcgttagctggctttgttccctgtacaatttcacttttggttattaaaatattcactgtaggaaataaatttgtaacccatttctcatattacctacacacagaaaaacaaaatttgatatcctggggtttatttgctgagggcgcttcccataaaagcgagagagtgtgcgttgggaaatgtgtctggttaactcttttatggataaactttagtcacaatcctcccccgcccccctctcacccccagcaccctcccaacctcccgacttcccgcctctcaagggctggtgacctaatagcatttttcttcgtgcatattttggcgtcgccccatggcctggctgccttcgcctgtctgagttttttgaaattcctgcatgttcgccccagattaagccagtgtgtctcaggatgtgtgttccgttttgttctttccccttaacgctccctgtgcaacgtgtctggggggaggagggcagggacgggagagagggaggggcagaggcgaggagctgtccgccttgcacgtttccaatcgcattacgtgaacaaatagctgaggggcggccgggccagaacggcttgtgtaactttgcaaacgtgccagaaagtttaaatctctcctccttccttcactccagacactgcccgctctccgggactgccgcgcggctccccgttgccttccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaacctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacg

SEQ ID NO: 2 - DNA, 1953 bp, Homo sapiens
tataaattccacgcaggcattgaattgaatttgttcttaaccaaatgcgttttatctatacctggcaggaatctagaagtgaaattacaagatttatttcattttaattctattatgaagcatttaatcacaaataccctgaaaatgaaaagataatttatcattttaccttgactgagcaactctcctcacttcacattcatgaatccataacgcagagaggagactggatgattaagtgtttgattagagaaaacagattaacctagcaaacataataaatttggctcataagcaggatggctttataaatgctcacaatacctctcctgtataaaatcatgaaccacttcctacagtgatgactccatcgaaatagttgagaaacataaagcaaatgcatgtttatggctttctctttgagacattaaaagggtattgaaaggcatatctgattcagcttataactctggatatatattaaggaacatgtaagaaaatattaatgcataaaaaaagctacaacttctcaagtgttctagtttccactttgtcaataattacgttttcaatgtccttctgtggactgtttccaaaggtgccaatccagacccaaagtttcagatcactcagattcacccttaaccttcataacacaacccaatagctttacgaaaaaagttgcatatttaggtagttgttatcccattatgacaaaatacataaaattagcgagatattttttagccttcaaataagtgggaaaaaatccttttagctgagattccatttacatcagaataaaaatctaagttatgactaggttgaagcaacgtcctgtgcagcgctccataaagttcacttagtcttcaagggttccttacttagctaggttagtattcctggcctctttttttagcagtgagaaaaaggatactctccctgccccagctttatttttaaactcacagccatatcctggaggtctctgctggctatttggcgcgtgggggcggaggggggccgggggaggggggcggggcggggtctggaggtctgtgctggctatctggcgtgtgtgtgtgtgtgtgtgtgtgtgtgtgtggttggaggtctctgctggctatctggcgtgtgtgtgtgtgtggtgtggtgtgtgtaagcagtgaggttgttttagggccagtccttcctccgccactttgctgactcaaagacccagaggctttcttggggtgcaggtaccatgattccttgggccctaagggaatttttgttaggctagaagagtgggtgtactcatgatgggtgtacccgaacattcctgggctcaacaaaaccgattatctttataaccgcggcgcctagcacagcgcctggtgccctaaacgttggctgcgggaacgtccgagacgcgggtgcggagccgggggcggaataactggttgcgcggcgctttgaccgtaggcgctggagcgcgtgcgttgcgtgcgcgcgcggaggcggctgcgtcggggcgcgagaaggtgcagttccccggcgggcgggcgggcgggcgggcgaagctgggctcggggccaagcgaggtctagccggagcgactgtgccccgcctcctgggcggagcgggcggctccccatggtcagagcctcgtgccggctcggcagcgcccggacgccgagcccagcgcgtcggccccccggcgtgcgggcgtctcagagccgcggaggggccgccgggaccgtttcagcgtggcggcgctggtgctggcgttggccctggaggacggccccgagtgatggctggcgcctgcctcccgggtgtctcccgggtacagatggagtcgtcccgcggccgccggcggcaaggtcggcagctgcgaggccaagagagaccccaggacacacacagctgcctcccggtgcgagaagaagaccccggcttgagagtgag

SEQ ID NO: 3 - DNA, 889 bp, Homo sapiens
cggccccatggctccgtgtcgtgtccaagggatgggctggcacctcttggaccaggcttaccaccagggcccttctctgaagccccagtctgaccggcctgctgctgggaatccccctctgcccccacactaacctctgctggggctgagccagggcgcgtcggacagtcagggcgacccagccagggcgaccgttggccccgctcctatggggcagcagggaccgacgtcagcagggtggggcgggcacccgagtggtatgccccgccctgccccgcctgcccgccctggtggccgtctgggggcgacaagtcctgagagaaccagacggaagcgcgctgggactgacacgtggacttgggcggtgctgcccgggtgggtcagcctgggctgggaggcagccccgggacacagctgtgcccacgccgtctgagcaccccaagcccgatgcagccacccccagacgaggcccgcagggacatggccggggacacccagtggtccaggtgtggcgggggtgaggggagggggggtgggagcggtggagatggggccgtggggagggagctgagatactgccacgtgggacgatgctaggtggggagggctgagctgggcgggctcctctggctgtggggccccctgtgttccttgtgggaggtggaaggaagtgagtgccctgtccttcctccctgccatgagattccaggaccggacctggcaagtgccctatcccagccagtgttcctggggctcttccaggcagggctatgttccccaggccaggggcattgtcctggacagtcaggaggcatacccctcgccaggtggaaccaccctgtgtatgcatgaccctgacaagcaggcgccaggacagtcaggaggccag

SEQ ID NO: 4 - DNA, 863 bp, Homo sapiens
gttgttgggtgtgaatggagaactgtgggccctccccgacaccttccagcgggacggcaacgggggcccagggggtgggcgccatcaaccccgtcccaccgccaggacggcgcgggggagggccggcgggggcggggcgtcctgtaaggcgcggcccccacccgcgggcggggcggcattcctgggaggccggcgctctgacgtggacccgggggccgcgggcacggcgggggggcggcggtccgggggcttcttaaaccccccgccccggcccagcccgcacttcccgagcaccgctccgaccctggagggagagagagccagagagcggccgagcgcctaggaggcccgccgagcctcgccgagccccgccagccccggcgcgagagaagttggagaggagagcagcgcagcgcagcgagtcccgtggtcgcgccccaacagcgcccgacagcccccgatagcccaaaccgcggccctagccccggccgcacccccagcccgcgccagcatgatgaacaacagcggctactcagacgccggcctcggcctgggcgatgagacagacgagatgccgtccacggagaaggacctggcggaggacgcgccgtggaagaagatccagcagaacacattcacgcgctggtgcaatgagcacctcaagtgcgtgggcaagcgcctgaccgacctgcagcgcgacctcagcgacgggctccggctcatcgcgctgctcgaggtgctcagccagaagcgcatgtaccgcaagttccatccgcgccccaacttccgccaaatgaagctggagaacgtgtccgtggccctcgagttcctcgagcgcgagcacatcaagctcgtgtccatag

SEQ ID NO: 5 - DNA, 2198 bp, Homo sapiens
aagagaggcacactccctctaccacaccgagggagggggcgttgagctgagaaaggttgagagaatgagggacccaggtaggtggacatcggccaagaaaggaaccacagcgggaggtaagaccgagagtccccagcttgaagcgtcaccactccgggattcccagattccaacgcgagcctggggaaagcccacagtggagagagtccggctggcagggaatggccctacccccggggtgaaatctcggagggtcgtgcagccgagtcgcgcctctgcgctgatgcgtgagagatgccggacgtcgcgtttgcctgtgcgagcctcgcggatgctgtgcagtcttggtcccctctgcgtgtgtctaacgccgaatgctggtgtctcgaggtgtgagcttcggggccggtgtctttaaagaaccaaagattcttaaggagtgatgatctgggtagagcggcccgacgtagccgcgctcccaggtctcggtgcgagtcctgcggacagaccagaggagacctgctggccagatgccccgggcccaaggcggacgccagactgtctctgcgccagccgggctggccttcggaatggatcaggcacccgggaggccggagtggatctcagaccctcaagccgggaacaaacccgtcgatgcccgtgggcctggagtccgcctcctccttcccgccccacccctacccctgcctccgaaaggcttcttcgctggtcagtagctgcgtgcccgtctgcctgaggctgggtcagaattggcgggctggtaacgaccccgtgcacaagcggctcccagtctctccagaaagggccgatgactaaggggtgggggtgggggcggagggctggaaggtgttagggaagaacgttagcggcctatcctgtcttcagcagcgccctctcatcttctagctctgacgccgagcagagcagttggagctcgggactgggaactgctggaattcctatttagacttctagacagtctagaaacaagaacctttctttccctgggcctcagtttccttgtctgtaaaatcaaaaggcgggctctaggtgtaggccttcttttcgcttggtgattctggattcctttccttggatccgtggggagggggtggcagcaacagtccagggcgttggccgtcctgtgcctcaagtacgtagtccccgtgcccgccccctcaacacccccagcagcccgcccccctaagcccgcagagcagggagctgagtgggaggggcagaggcggggccggttcccagtccctgctggcggactagagtggcgcgggctgagcgtaaaacctgggatagccactcccccttttccttatccccgcccccctgccattggctcccgggagaggttgacatcaaagccgcggtcttatataagccagatccgcaggggagtccgcagaagggttaaacaggtctttgggcttcggcgacctcgcccgcggcagaaaccggtaagaagacagtgggctgcgcgtctcattttcagccttgcccggactctcccaaagccggcgcccagtagtggctccagagcccacaggtggcccccggcagtctctggggcgcatggagcggcgttaatagggctggcggcgcaggccagtagccgctccaacatgaacctcgtgggcagctacgcacaccatcaccaccatcaccacccgcaccctgcgcaccccatgctccacgaacccttcctcttcggtccggcctcgcgctgtcatcaggaaaggccctacttccagagctggctgctgagcccggctgacgctgccccggacttccctgcgggcgggccgccgcccgcggccgctgcagccgccaccgcctatggtcctgacgccaggcctgggcagagccccgggcggctggaggcgcttggcggccgtcttggccggcggaaaggctcaggacccaagaaggagcggagacgcactgagagcattaacagcgcattcgcggagttgcgcgagtgcatccccaacgtgccggccgacaccaagctctccaagatcaagactctgcgcctagccaccagctacatcgcctacctgatggacgtgctggccaaggatgcacagtctggcgatcccgaggccttcaaggctgaactcaagaaggcggatggcggccgtgagagcaagcggaaaagggagctg

SEQ ID NO: 6 - DNA, 1945 bp, Homo sapiens
ctggatgacagagtgagactccgtctcaaaaaaaaagctccatttgggaggccgaggagggtggattacctgaggtcaggagtttgagaccagcctggcccacatagggaaaccccatctctactaaaaatacaaaaattagtcaggtgtggtggctgacacctataatcccagctacttgggaagctgaggcagggagaatcacttgaaccggggaggtggaggttgcagtgagctgagatcatgccactgcactccagcctgggcgacagggtgagattctgtctcaaacaaacaaatttaaaagctccgaatcctccaaaaataccaagattttcctgtcggtaactagagatgggtactgatgattatttttaataggtgattttcaaagatgtgaacgttatccatggagatttaagtctccaaaaggaaaaaaaatgcatacctttatactaaaacttcatcaccagtcaaatttggatcatcactaaattggcttctacacctctctcctaatataaggtacttgtgtaagtttgcagttgtgagacacttatttcctcatttttaatgtcttctcagtagggccactgatatagtcactatttgactgaccagaatggttggcactggtgattggctcataaagtgccctcgatttagggggctcaattatcaaaggtttaaatcctagcccaaaccattgctgtgatgggggttaatcaatgaaccactcagcttcacttgcaaaagcgggatcacaatagccgctttcgtcatgacccagcctaggtgagatttagtacttaagtacactgccaggcacacaaggttaatttaacaatttaacacatttgtttcctcatccatttctccaaaccttccaactaatcctaacgttcgttcggccaaatgggccaggaattcacttaaacaaaaacaaaaaacaaaacaaacaaaaaaacactccctggggcttggggaaggaggcaccgccgcccatgtcgcagtctgggggtggctcagtcctcagcacccagatctacggccataatgctcttcgaggccaaggagcccggatgcggggcgttgccgaaggcgtcttgctcaggctgcgggaaaggagaggggtgggagcggggtgggggcatcgcgacccagggcaaggcggcgagtcgccgtcttcgagtcccacctgtccgaagcggggtgagaaaaggcaaaacatggcaaagccatgcacctcccagggtgggcaactcacggccggtgaacgccggacccttagcagtttccagacctttggaaccggaagcggagcctgagagcgcgcccgagagggcgtgaacgggaccgctttcccggaagtgcttgcggcctctgcccagcgagctgccccggggtctctctggtttcctaatcagggcaacgccgcgggagagaacctttaccttggctgcactaagttctcggtgccactccctggcagggcgggaccttgtttaggccctgtgatcgcgcggttcgtagtagcgcaaggcgcagagtggaccttgacccgcctagggcgggaagagtttggcccgccgggtcccaaagggcagaatggacgggctcctaaatcccagggaatcctctaaattcattgcagaaaacagtcgggatgtgtttattgacagcggaggcgtacggagggtggcagagctgctgctggccaaggcggcggggccagagctgcgcgtggaggggtggaaagcccttcatgagctgaaccccagggcggccgacgaggccgcggtcaactgggtgttcgtgacagacacgctcaacttctccttttggtcggagcaggacgagcacaagtgtgtggtgaggtacagagggaaaacatacagtgggtactggtccctgtgcgccgccgtcaacagagccctcgacgaag

SEQ ID NO: 7 - DNA, 2379 bp, Homo sapiens
aagcttgtggtttacttggacctctgcctcatctttcttcttttgcgcttcagcctgcgcattcgcttcctccactaggctctcatggtgcagaggtttccaagaagatggtgtgaaggccgagatcatttggttatattataaaatagaatgcaaattcacacaagtttttgttttttatttatttatttttttagagatgaggtcttgctatgttgtttagtctggtctcgaactcctggcctcgtgatcctcccaccttgacctcccaaagtgctgggattacaggcctgaggcctgagccactacacccaactgaattcacatttttttttttcttttctgagacggagtctcactctgtcacccagtatggagtgcagtggcgcgactgcggctcactgcaagctccgtctctcgggttcaagtgattctcatgcctcagccccccaagtagctggaattacaggggtgcactaccacacctggctaatttttctgttttagtagagatggggtttcaccatgttgcctggtctcaaactcctgactttaagtgatccacacacctcagcctcccaaagtgctgggattacaggtgtgagcctccacacccggccgaattcacatgaattttaaagtgatgtcttcaaagtggtttcactgtggggatgggcagctttttgttatacatctagaacgttcctcttctgtttctatgaatactcggttggaaagggctgaaaaacggtcttaagagattatctgattcgtttcccagttttattactcacatatcagctgtaatttgagcacgttttctgattgagacaagactcagatggtattaaacattactacaacacatccgggcacggtggctcacgcctgtaatcccagcactttgggaggccgaggcgggcggatcacgaggtcaggagatcgagaccatcctggctaacacggtgaagccctgtctctactaaaaatacaaaaaattaggcgggcatggtggcgggcgcctgtagtcccagctactcgggaggctgaggcaggagaatggcgtgaacccgggaggcggagcttgcagtgagccgagatcgcgccactgcactccagcctgggcgacagagcaagactccatctcaaaaaaaaaaaaaaaaaaaaaaaactacaacactataaattcatatctattataatagtactttgtgcagggccctaccctaagtccttaaccgaacccggaagcgagaagatgacttttgtttgtttttagagatgggcgcctggctctgtcgccagcctggagtgtgggggcgcgatctcgactcacagcagcctccacctcccgagttcaggcgatcttcctgcctcagcccctcgaggagctgggaccaccggcgcgctccatcgcgcccggctaggagctgactttgaatccgggctctgcgcctggccttctgcatctctataagggaagacatctgtgacctcggggcaaaggtcaaattagatcctgggtaggatcctgttcccgctgcccctcgggctggcactgccaggagtactcagagctcaaagctgggatctgcagtcccttacccactcagtgcacgccgcctaaggctttgcgcttcacctttactcacctcgaagccctggacatccgcatctgccctaagacttctcacctcagtagcagaaggaagtcgcgtcagctggccacagcctctctcctaggagaccgtccgggaaaagcgagtcagggtagaccctgaggcccctcagctccggctattttcagatctgtcgctccttcaccctcagcctttcaaacaggccactccaaaaaaaagcccaatcacagccttccttcttctcctggccttccggcactgtccaatcaacgtacgccatctatcggttagtggtgttgcggggccacccttcccgctggtttccctcgtggtgtgtaaaggcagagaggaaaggcgaggggtgttgacgccaggaaggttccatcttggttaagggcaggagtcccttacggacttgtctgaggaaagacaggaaagcgccagcatctccaccttccccggaagcctccctttgccaggcagaaagggtttcccatggggccgcccctggcgccgcgcccggcccacgtacccggggaggccgggccccggaggacgagggaaagcaggccgggcgccgtgagcttcgcggacgtggccgtgtacttctctcccgaggagtgggaatgcctgcggccagcgcagagggccctgtaccgggacgtgatgcgggagaccttcggccacctgggcgcgctgggtgaggccgggccctccggccgggacccccagtccgtcg

SEQ ID NO: 8 - DNA, 933 bp, Homo sapiens
gagacgtactctggctctgtcgcccaggctggagcgcaatggcgccatctcggcgcactgcaacctccacctcccgggttcaagcgattctactgcctcagcctcccgagtagctgggactacaggcgcgcactaccaagcccggctaatttcttttgtatttttagtagagactgggtttcacgatgttggccgggctggtctggaagtcttgacctcaagcgtgcgccctctccgccactgggtaaggcggggggcggaatagggggcttgcaatttcacactagaggcgggcgccgtgggggaaagaagagtcacgtctcccacggttcgtagaggaaggcctgcctgagcctggagcgggggcgggagagccacagtttggcatccccagggcatcccccagcccgcagactaccaggcctccagaggacaggaccccacccccggccacaggccctgcccccagcactccccgcaccccgcctccaagactcctccgcccactccgcacccaacttataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcggctcggccaggggctgcgcgaacacgcggagcgcacccgcagccagctgagcgcgctggagcggcgcc

SEQ ID NO: 9 - DNA, 6096 bp, Homo sapiens
atctgcacctcctcatatagggttgatccaagtttcacagacatcactgagttcttagtggactcagctattggggctgttctcacactttttttttctttgcaagaatcagcaatgggtgcaagtggacctgtgtaggacgtccagtgaaacattgtgttggtgaatcagctagaatccatccaagaactcagccagcctggtgtggggtgagatctgatccttgaatgtccctcagtggcttttagggctggcaggttcagaagggccctctcatcacccccccagggcctcattccttgtttaacactttgctatcacagtcttgaatccttgtaattgaacaatggaccccacattttcactttgcactggtttctgattctgtaaccgatcctgtccccctctcttgtctcattcactctgggaattgtccccacattctgagacctttcagcagtgccccaacgaggttcctgcccttatctgaagctccaccctcacccccatggcggcaccgcaggcagccctgcttttgcgtcccgcgtaggcaggctgtgcaccggagtcacgaccccctgattcagcctaggcagccacagcttgactgctcccgccggacaagccctactgtgctatctgccgctcttcccttcctcttcccagggggtccgcgtcaggggaggcgcagctgtgtgcattccgggagcttcagacccccgtgtccagcagctccttcgtttcctgggtgctggggcggccttcccagcgaagagctcaactcagcgggacgtttggaggctctctgccccaaggcgctggggagtgtgcggcgggacagtcgtgcttgcctttttcactttcagagtgtccacgccccacccgtttggtcactgcaggtcagtccagtccagcccggcccaccccaccggtgcgtgtctgtcgcacgtggcagacgccatactctctgttcttgtttaaagcccaggatctactgggccctggaggcaagaggtgaacgcagcggaatccacgctgagctgcccgggaacggagcttccaaccccagaaggaggactctgtgctcctacaccttaaccctttttagcccgaaacttctccaacttccttggctttgtttagagctcgacagcgccgccccctggcgctcgttgtgaggacagtagaggagagaggcaagggtgtttttaaacagtttgcctctcaccattatgggggcgacccgagggggagacccactcttccgcattcccggtaagtgaaccaccggaagaggtcgaaagtgacggattcccatgtcctcctccagccccccccccaccctgcccatccacaggacggtggctcttcagtgccctttgccgagcaagtggcgtttctatgcacgtgggtatcaattcggactctggacgaaatggaaacctccttagccgacccgggtgggatcagctgggatcctgcgcgctcccctggggggttgccagccactctgttggggtgcaagaagcaccatccttcggaagctgggccgaaactggccaggctgactcgctcccacgcgcccgcccctacccggcgccgcagcaattcacctgccaccgcctctgagccgggtccggacttcggcgccctgacagtgtccccgcgacttccccacccgatgagatggggtctggcgttggccagtgcgtgtccagggactcgcgggtccctggccagccatggggcagagggcgctggtgttaggccagtcttccccaccctgccccgtcaccccagccacacccactgtcctgtgaggccaagcgcgctccgctggtttcctgagccaggcaccttggccgcggacaggatccagctgtctctccttgcgatcctgtcttcggggaagtccacgtcctaggcaggtcctcccaaagtgcccttggtgccgatcacccctcccagcgtcttgcaggtcctgtgcaccacctcccccactccccattcaaagccctcttctctgaagtctccggttcccagagctcttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggccttccctggcgcctgcccctgtcggccccgcccgagaacctccctgcgccagggcagggtttactcatcccggcgaggtgatcccatgcgcgagggcgggcgcaagggcggccagagaacccagcaatccgagtatgcggcatcagcccttcccaccaggcacttccttccttttcccgaacgtccagggagggagggccgggcacttataaactcgagccctggccgatccgcatgtcagaggctgcctcgcaggggctgcgcgcagcggcaagaagtgtctgggctgggacggacaggagaggctgtcgccatcggcgtcctgtgcccctctgctccggcacggccctgtcgcagtgcccgcgctttccccggcgcctgcacgcggcgcgcctgggtaacatgcttggggtcctggtccttggcgcgctggccctggccggcctggggttccccgcacccgcagagccgcagccgggtggcagccagtgcgtcgagcacgactgcttcgcgctctacccgggccccgcgaccttcctcaatgccagtcagatctgcgacggactgcggggccacctaatgacagtgcgctcctcggtggctgccgatgtcatttccttgctactgaacggcgacggcggcgttggccgccggcgcctctggatcggcctgcagctgccacccggctgcggcgaccccaagcgcctcgggcccctgcgcggcttccagtgggttacgggagacaacaacaccagctatagcaggtgggcacggctcgacctcaatggggctcccctctgcggcccgttgtgcgtcgctgtctccgctgctgaggccactgtgcccagcgagccgatctgggaggagcagcagtgcgaagtgaaggccgatggcttcctctgcgagttccacttcccagccacctgcaggccactggctgtggagcccggcgccgcggctgccgccgtctcgatcacctacggcaccccgttcgcggcccgcggagcggacttccaggcgctgccggtgggcagctccgccgcggtggctcccctcggcttacagctaatgtgcaccgcgccgcccggagcggtccaggggcactgggccagggaggcgccgggcgcttgggactgcagcgtggagaacggcggctgcgagcacgcgtgcaatgcgatccctggggctccccgctgccagtgcccagccggcgccgccctgcaggcagacgggcgctcctgcaccgcatccgcgacgcagtcctgcaacgacctctgcgagcacttctgcgttcccaaccccgaccagccgggctcctactcgtgcatgtgcgagaccggctaccggctggcggccgaccaacaccggtgcgaggacgtggatgactgcatactggagcccagtccgtgtccgcagcgctgtgtcaacacacagggtggcttcgagtgccactgctaccctaactacgacctggtggacggcgagtgtgtggagcccgtggacccgtgcttcagagccaactgcgagtaccagtgccagcccctgaaccaaactagctacctctgcgtctgcgccgagggcttcgcgcccattccccacgagccgcacaggtgccagatgttttgcaaccagactgcctgtccagccgactgcgaccccaacacccaggctagctgtgagtgccctgaaggctacatcctggacgacggtttcatctgcacggacatcgacgagtgcgaaaacggcggcttctgctccggggtgtgccacaacctccccggtaccttcgagtgcatctgcgggcccgactcggcccttgcccgccacattggcaccgactgtgactccggcaaggtggacggtggcgacagcggctctggcgagcccccgcccagcccgacgcccggctccaccttgactcctccggccgtggggctcgtgcattcgggcttgctcataggcatctccatcgcgagcctgtgcctggtggtggcgcttttggcgctcctctgccacctgcgcaagaagcagggcgccgccagggccaagatggagtacaagtgcgcggccccttccaaggaggtagtgctgcagcacgtgcggaccgagcggacgccgcagagactctgagcggcctccgtccaggagcctggctccgtccaggagcctgtgcctcctcacccccagctttgctaccaaagcaccttagctggcattacagctggagaagaccctccccgcaccccccaagctgttttcttctattccatggctaactggcgagggggtgattagagggaggagaatgagcctcggcctcttccgtgacgtcactggaccactgggcaatgatggcaattttgtaacgaagacacagactgcgatttgtcccaggtcctcactaccgggcgcaggagggtgagcgttattggtcggcagccttctgggcagaccttgacctcgtgggctagggatgactaaaatatttattttttttaagtatttaggtttttgtttgtttcctttgttcttacctgtatgtctccagtatccactttgcacagctctccggtctctctctctctacaaactcccacttgtcatgtgacaggtaaactatcttggtgaatttttttttcctagccctctcacatttatgaagcaagccccacttattccccattcttcctagttttctcctcccaggaactgggccaactcacctgagtcaccctacctgtgcctgaccctacttcttttgctcttagctgtctgctcagacagaacccctacatgaaacagaaacaaaaacactaaaaataaaaatggccatttgctttttcaccagatttgctaatttatcctgaaatttcagattcccagagcaaaataattttaaacaaaggttgagatgtaaaaggtattaaattgatgttgctggactgtcatagaaattacacccaaagaggtatttatctttacttttaaacagtgagcctgaattttgttgctgttttgatttgtactgaaaaatggtaattgttgctaatcttcttatgcaatttccttttttgttattattacttatttttgacagtgttgaaaatgttcagaaggttgctctagattgagagaagagacaaacacctcccaggagacagttcaagaaagcttcaaactgcatgattcatgccaattagcaattgactgtcactgttccttgtcactggtagaccaaaataaaaccagctctactggtcttgtggaattgggagcttgggaatggatcctggaggatgcccaattagggcctagccttaatcaggtcctcagagaatttctaccatttcagagaggccttttggaatgtggcccctgaacaagaattggaagctgccctgcccatgggagctggttagaaatgcagaatcctaggctccaccccatccagttcatgagaatctatatttaacaagatctgcagggggtgtgtctgctcagtaatttgaggacaaccattccagactgcttccaattttctggaatacatgaaatatagatcagttataagtagcaggccaagtcaggcccttattttcaagaaactgaggaattttctttgtgtagctttgctctttggtagaaaaggctaggtacacagctctagacactgccacacagggtctgcaaggtctttggttcagctaagctaggaatgaaatcctgcttcagtgtatggaaataaatgtatcatagaaatgtaacttttgtaagacaaaggttttcctcttctattttgtaaactcaaaatatttgtacatagttatttatttattggagataatctagaacacaggcaaaatccttgcttatgacatcacttgtacaaaataaacaaataacaatgtg

SEQ ID NO: 10 - DNA, 2500 bp, Homo sapiens
acccacttctgtgtgtggatagtatcctgcaggagagatgttgtctgcagtgtgagctgggcccaccggagtgtgtgaataggatcctgcaggagaaatggaatccggagtgtgagctgcatccgctgtagagggtggataaaatcctgcaggaaagatggcatctggaatgtcagcgggagccaccgacctctgaggatgcaccccgcaggtgtgatgcggggccagttccaaggctgggttaggttttaccctggcttctgtgttgtactctcattctcttcctctttcttctaatacctgctctgggaggcatcaggccatgtccagtgtgcaggccatggagacccacacggcaaggaactggaaccccctgccagcagcctcgggggtccagtccttagatggtgccctgtggtcagcaatgcacctgtgacctccgggctatgtctcgtggtagttgcttttgtgttttaacatagcaacaggaaactagcctattacccaccaatcccattccaggctgctttcaaacgcagctcaggctagaacaccagcacggggacacagctgagacttggggtttgcgacgggaacacgcccatgctgtgcctctgaatctggcaccgtcaccctgtggcctgggttcagcaacttggcctcaccttccttgtctgtgaaattcagactgggtccttgtgagatgattggagagaatgtatgaactatgtgagaacgccacctttgtgcgtatctcacgcagtgtcttccctcctttccaaagtcttctgctgtctctagacacacccgacgtggggggggggggttccctgggtctcctcctaggtctgtcccaggagggcacgcactgaaggccgcgagaatcccgggggctgcattgcgccgcgccaaggactccacacaggacctttcattttcccaactgtgctgagccaggcggccggcagagagcaggtggctgacaggccccggggagccggaccgcctgggtctaatcttcccgcagactcccttgctgtgcgctttggggcttgggcctcagtttcctcaaaaggaatgaggggcttttttggaacgttaaataatttcctacgtggttgcgggtagggagaaggagaaagagaggagcgcgcctgcgcgcctggaatcgtgcccggatcagagcaagcgctctaaaagtgttacaaacattaaggcgccaactaaaaaacccgtagtgagcgcaggcagaaaccacgggtaagagaagtggagaagcttcgcgtaggccccagggtcccgagccccgagtctcgagcgcagaatcaggggtgccaatgctctcctccgcgcccccgagcgctcgccttggccatgcgggccgccccaccgggatgagggcgctcaggccggacgctggggccccgggttctcgccccgccccgccctcggggattcagaggggccgggaggagcctcgcgcatgtgcacagctggcgccccccgccccccgcgcacagctgggacgtgggccgcggccgggcgggcgcagtcgggagccggccgtggtggctccgtgcgtccgagcgtccgtccgcgccgtcggccatggccaagcgctccaggggccccgggcgccgctgcctgttggcgctcgtgctgttctgcgcctgggggacgctggccgtggtggcccagaagccgggcgcagggtgtccgagccgctgcctgtgcttccgcaccaccgtgcgctgcatgcatctgctgctggaggccgtgcccgccgtggcgccgcagacctccatcctgtgagtgccgcgggggacgccgggggcgcggggtccggggcttcgtggagatccgggagcgcaggggtgatcggaggtggggggcgcggagggtggagggggcatcgggcgcgcggggggcctggggacttgggacgcagaagggaacctccgaagggggacgtggggggacctgggcgcggggacccgctgggcctttgttcgccctgcgggagacgccgaggggcggaacagagcgctgtgcgcgcggccttcgtagccgcctttgttcggaactcggaatccccgcaggactgggaagttgttggagcctccggggctccccccgctcgcctcccgccgccccctctcatgctccgccggcctcccgcttccccctggttcgcggcccctcctccgctcacctttcccccgctcaggacccctcggtccccctccgctccccgagcgcggcgcagccccctccgtcctcccagccccctccgccccgttcctcgtcctgttcgctcccctcctccgctcctcttcctcctccccttcctcctcctcctccccttcctcctcctcctccccttcctcctcctcctcccttcccctcctcctcccccccttccttctcctcccccagcctccgccctctccccctcccccgccccttggagcgcagtgcccaccccatccccccgcgccgg

SEQ ID NO: 11 - DNA, 2200 bp, Homo sapiens
cctcgccccctccagccggccccccgggcccctcctctcggcgcccggaccttggccctccctctcctttcccacttctctctttgccctaacttcgcccccatcccccgctcatttcctctcgcacccgggctcgccaatccctctttccaagtccctcttccagcccggccttcctctcgggttcgccccccttctccccaatctccgtcctcttccctcccttcgccctcccccccttccttcctcttcccctcacccaaccctggttcccctcgttcctcagtcccgatctctcccttactctgtccccgcccactctgcgccggcctctcagtccgggttgagccccacgtgtggacggccgcgcccccactgacagccgccgcccgccggcccgccccgcgccccgccgggcctctaaaacccccgcgccgcgccctccaccgccgcatcttctccagcgcccagcctcccgccctctctcttgctggccgcacgccccggccccgcgcacctccgcccggctccgcagccgctacccgcgcttcgttgccctgtgggactccgagcgagcccggagggaaccctcctcttcttctgggggcgacttttgtttgcttgcctgtttctttctggtgacttttgcagctttccaatatccgtcttcggagcgcacgggaatccgccgagctctgcgtgcaggcccttttttcttttgaggttcacattttttgaaattttacgccagggcttttgtaatttcctcccccgcccgctgacggtcctggagtcgctcggggctttaggccggttatgcaacgtgtaccgctcggggctgccggctgcacctccgccgcgcctcgccgctcactgcgctagacccggcgccccgcgtctcgcttcgcgggcagtcagggggccggcgctctgtcgaggtctccagctagagcagggagcccgagcccgagggagtccccggagccgacgaagggcttattagaccctgactcttttctgaggcgcgcagattttgtctttgatcactccctctccgcgggtctacggccgcgcgctttcggcgccggcgatggggagaagacggaggctgtgtctccagctctacttcctgtggctgggctgtgtggtgctctgggcgcagggcacggccggccagcctcagcctcctccgcccaagccgccccggccccagccgccgccgcaacaggttcggtccgctacagcaggctctgaaggcgggtttctagcgcccgagtatcgcgaggagggtgccgcagtggccagccgcgtccgccggcgaggacagcaggacgtgctccgagggtaagtgggcaagcggctccgcacctagggctccggcttgggggaggggggaatcctcagtttggcggctttctggcccactccgtcccagaccctttagctggagcctagagctgcagccccctttgccagaatatccaaagacccccaggagcgcgtcccccttttccttcccaaccccgcagctcagcgggcggaaagccctctctccgggggttgggcggcgggtggttagggggtccaggggtgccgatcgcagagcgtgtgcagagctcgcgctgcgggaacaggttctgaatgtccggcggcaggcgggcctgggtccgcctgctgcaggggccagagaagcctgcttgctccccacgtcggggccgccgctcgtgagccttttgtttgaggacgtgtgcagggttcacagctcaccttctcatcgtcaacccgagcgctccaccttgcgacgcgctttccttgacacgtcggggccaaagtaacagttgaccaaggaggaatggatttgggaaggagggcaaggattctttggaacggaatggtccctttgttctctgcatctggaagctagaatagtagcaaattatatgtttccatgcctcttttcgccctttaaaaaggcaggcaagggacgacagatgaaaggcagtgtttagacatttctgaccctcctgcattccagcatctagctcttttgcttccacgtctgcctcccgatctccaataatttgaagtgtaattttgatttgtttgttgtcctgaaatctactcgctcggggcattgcttacgaagaccgtttatatgttgctgcatccctctacctatctgttacgtgaccgcgcttgt

SEQ ID NO: 12 - DNA, 2000 bp, Homo sapiens
ttggaagaaaaggatctccgaggaaggggctgagagaagggcagggtgaactggactaaaggccagagtaggaaggagaagaggggccaaaaaagaaggggatgaaattaagcacagaagatgggtaaagaaaaaagtatcagggaaagggcaaaataagagaaagccttgaggataagagggtagaaggctaaagaacaaggggaccactgggtcggggaagcgctgcctgaacggcgggacagtgacaaagaaagggcgctggcgatattcgcaccaagggtgcgaaacgcaatcgggaggtgagaaatggaaagaaggcgaatgcccggctacaagtagcctgggactgaaaggggacctgggggaggggctgggcccagggcagaaaagtccaggttcccatgcggcctgggcccacgtggagcgggcgctgaatcaccgttcagccgcccccctcccctcctccccgaccggtgcccgcagtccccgcctcctcggccgccgcctccacggggcggggccctggcccgggaccagcgccgcggctataaatgggctgcggcgaggccggcagaacgctgtgacagccacacgccccaaggcctccaagatgagctacacgttggactcgctgggcaacccgtccgcctaccggcgggtaaccgagacccgctcgagcttcagccgcgtcagcggctccccgtccagtggcttccgctcgcagtcgtggtcccgcggctcgcccagcaccgtgtcctcctcctataagcgcagcatgctcgccccgcgcctcgcttacagctcggccatgctcagctccgccgagagcagccttgacttcagccagtcctcgtccctgctcaacggcggctccggacccggcggcgactacaagctgtcccgctccaacgagaaggagcagctgcaggggctgaacgaccgctttgccggctacatagagaaggtgcactacctggagcagcagaataaggagattgaggcggagatccaggcgctgcggcagaagcaggcctcgcacgcccagctgggcgacgcgtacgaccaggagatccgcgagctgcgcgccaccctggagatggtgaaccacgagaaggctcaggtgcagctggactcggaccacctggaggaagacatccaccggctcaaggagcgctttgaggaggaggcgcggttgcgcgacgacactgaggcggccatccgcgcgctgcgcaaagacatcgaggaggcgtcgctggtcaaggtggagctggacaagaaggtgcagtcgctgcaggatgaggtggccttcctgcggagcaaccacgaggaggaggtggccgaccttctggcccagatccaggcatcgcacatcacggtggagcgcaaagactacctgaagacagacatctcgacggcgctgaaggaaatccgctcccagctcgaaagccactcagaccagaatatgcaccaggccgaagagtggttcaaatgccgctacgccaagctcaccgaggcggccgagcagaacaaggaggccatccgctccgccaaggaagagatcgccgagtaccggcgccagctgcagtccaagagcatcgagctagagtcggtgcgcggcaccaaggagtccctggagcggcagctcagcgacatcgaggagcgccacaaccacgacctcagcagctaccaggtaggaaccgcggctgcgcggccagcctgcgccagcgccagcgccgcgcgcccccgacacttgggctcgtgcccaggcgccctctccgccgcgctccctggtggccgctcgctagagcacgcgcgccgcagacctagggtatttgcggatcagcgtcctcgcccatctcatcctccacactccgcccccacccacctgccccagctgctaagggtcttgacctttttcagaaacgtgcatcttttccagttctaattttgcacgcttgcacgtttaaagcaggagggatgaattcggtagtggataa

SEQ ID NO: 13 - DNA, 2300 bp, Homo sapiens
tcagattgtcattgggagggtgaataaatgaatgcttgcattatgagagtttgggggcagaaatatgccacagactcttatctgaagccatcagatttagtggctgcgaacccaccgaagtcagggatttacattttttacagcaacgagagaaaacttcccctttcctctgcagaagtcaggactggatctcaaaaatagaaatgtgtcctcctaaatgtgtgcccatccccgtggttgacaaacaacggatttcccaagatagctgccacacacttggtttctaatctctgtattgcttccccgccagaatgtcgaagtccttcccgaatatgcccagtcatactttctgaacttttgagcaaacaccgtccggcttcttgtgctttcctcaaagaccccaggcaccggcagggaggacacaggccggggcagagcgcccctgcgcgggggattcctgccactccgcgccagcctgcggcgcaaacgctcttctcagccgcagtcccacccgctgctggcaatctgaatgaggagccgcgctatttttacctccccggctgcaatcctttatatttacatgcaggaagcaaatatataagggattaagaaggagatgcgtggccttagtttatccagagcaggaagaggttggaataggagagggtatgtgaagtctggggtggtggaaaaggcaggtggacttcggctggttgttttctcccgatcatccctgtctctggcctggaaacccccgtactctctttcttctggcttatccgtgactgccggctccccctccaccgcccccatcttttgaggtaccacccgtcacctccgatgctgcttgggctgctgcatcactctgctgctttacccccttccccgccccccaacaaagcatgcgcagtgcgttccgggccaggcaacagcagcagcacagcatccagcaacagcatcagcacccgaagccccgctcgggcgcgctctcggggggcggggcgcacgcccgctccgcgcgtccccgcgccgctcgctcccgcgcgtccccgcgccgctcgctcccgcgcgccgcctcagcatcctcaggcccggcggcagcccccgcagtcgctgaagcggccgcgcccgccgggggagggagtagccgctggggaggctccaagttggcggagcggcgaggacccctggactcctctgcgtcccgccccgggagtggctgcgaggctaggcgagccgggaaagggggcgccgcccagccccgagccccgcgccccgtgccccgagcccggagccccctgcccgccgcggcaccatgcgcgccgagccggcgtgaccggctccgcccgcggccgccccgcagctagcccggcgctctcgccggccacacggagcggcgcccgggagctatgagccatgaagccgcccggcagcagctcgcggcagccgcccctggcgggctgcagccttgccggcgcttcctgcggcccccaacgcggccccgccggctcggtgcctgccagcgccccggcccgcacgccgccctgccgcctgcttctcgtccttctcctgctgcctccgctcgccgcctcgtcccggccccgcgcctggggggctgctgcgcccagcggtgggtatggccccgtgccctttgcgttggctttcccgcggggccctgcagaggaaagcgaagggcgcgcgggtccgtgtgctccgggcttgtccccggctcggcctttccttccctccctgcctgtctttccacccttctcgttcccaaacccccattcatcccagttcacttttggaagtccatttctgttgcattcgcgaaaaacccattccaattcttgttggttccactgggaggtgtttagtggatcctgggtccctcagcgatctctgtgcaacttgcggaggggcaaccagtggatgggaaatacagcgagggagcaagttgctacttgcgtggtggaaccttaatgtgaatgcggggaggatgtagtgataatagtggtaatgggctgtttcctcaaatttcgtatccggcgcattcagtgcggttggaattaaggtgggggaggcacacttcggggaccaaagaattaaggtgctgaagacatacttcatgcacgacctttggttctgatttctcaaagtgcttgtcattataatgaacaattaatataataccatcttctatatattgatgattggaagtcactgaaagcagaaagctggctttgtcaggaaaataaaaagaaattgggaagctgccagcatctgtatccctacatggc

SEQ ID NO: 14 - DNA, 3000 bp, Homo sapiens
tactgccgactttaggtctctctggatctcaggcccccttctctaagatgcatcctagaggaccaaaaatacactttatttgggcttcgcctgcttttgtggaagggtagtttactagaggatataatctcgtgttttaatttgctctctctcctaaaggaaatgtggagaaaaaaaaaaagcagaaattggaaataaccaatatttagtttatttcattcgattcttaggggaactggtgaggagcctaagatgattttcccttcctagagaaagaatccaaagtccagggaaatagcgacaggggagttcaagactgcccctgctagtccttccttggctactctccgctgcgatcgcaggatagctctcattagcaggagaatcgggcaagtgtgtggataagtagagagtgtgttgaacaacttgtaacgttttatgaaatacgcattgtcatggttccctaaaaggctttgcggaagccgtttgtctttactaatcaagtctttacttacacaaaagtagaagtagaagtagttttagaaaacatactaacaatcttctatccccttgaagaccagagtagcagaaaacaggtgatttgcattataaaattgcactcactttttcctcctttcagatttcacattacattagcccatttgtgttacggtgtataaaaaatggaacaggcgcctccactgcattgttctcctttaaaaatagatcacttacaccctaactttgttttccttaaattcgattcttaacaggagagctttctattatttcagatggagtgaggttgcacgactgggatggaagaaaggaatcccttaaatttgggggaatttctgttctctgttccaagaccattttacttggggtgtgggggtgggcgcggcggtcagggcagtggaacgcagtcgcggctgcgccatccctgcacttccaggcgcgcgggagggaccggcggggacgcgagctgcggactctggcgaactcgggggaggcagacagggggaggcggacacccagccggcaggcgtctcagcctccccgcagccggcgggcttttctcctgacagctccaggaaaggcagaccccttccccagccagccaggtaaggtaaagactgctgttgagcttgctgttactgagggcgcacagaccctggggagaccgaagcttgccactgcgggattctgtggggtaacctgggtctacggaagtttcctgaaagaggggagaagggtttgcatttttcctatggaggattcttctctctctagcatttcgtttgatgtattcaactggtagaagtgagatttcaacaggtagcagagagcgctcacgtggaggaggtttggggcgccgcggcgccacccccacccctcctcgggaccgcgcctatttctaaagttacacgtcgacgaactaacctatgctttaaattcctctttccagccccgtgagtccgcggcgacattgggccgtggggtggctgggaacggtcccctcctccggaaaaaccagagaacggcttggagagctgaaacgagcgtccgcgagcaggtccgtgcagaaccgggcttcaggaccgctgagctccgtagggcgtccttgggggacgccaggtcgccggctcctctgccctcgttgagatggacaacgcctcgttctcggagccctggcccgccaacgcatcgggcccggacccggcgctgagctgctccaacgcgtcgactctggcgccgctgccggcgccgctggcggtggctgtaccagttgtctacgcggtgatctgcgccgtgggtctggcgggcaactccgccgtgctgtacgtgttgctgcgggcgccccgcatgaagaccgtcaccaacctgttcatcctcaacctggccatcgccgacgagctcttcacgctggtgctgcccatcaacatcgccgacttcctgctgcggcagtggcccttcggggagctcatgtgcaagctcatcgtggctatcgaccagtacaacaccttctccagcctctacttcctcaccgtcatgagcgccgaccgctacctggtggtgttggccactgcggagtcgcgccgggtggccggccgcacctacagcgccgcgcgcgcggtgagcctggccgtgtgggggatcgtcacactcgtcgtgctgcccttcgcagtcttcgcccggctagacgacgagcagggccggcgccagtgcgtgctagtctttccgcagcccgaggccttctggtggcgcgcgagccgcctctacacgctcgtgctgggcttcgccatccccgtgtccaccatctgtgtcctctataccaccctgctgtgccggctgcatgccatgcggctggacagccacgccaaggccctggagcgcgccaagaagcgggtgaccttcctggtggtggcaatcctggcggtgtgcctcctctgctggacgccctaccacctgagcaccgtggtggcgctcaccaccgacctcccgcagacgccgctggtcatcgctatctcctacttcatcaccagcctgagctacgccaacagctgcctcaaccccttcctctacgccttcctggacgccagcttccgcaggaacctccgccagctgataacttgccgcgcggcagcctgactcccccagcgtccggctccgcaactgcccgccactcctggccagcgagggaggagccggcgccagagtgcgggaccagacaggccgcctaggcctcctggggaaaccgactcgcgccccatacccgacctagcagatcggaagcgctgcgactgtgcccgcaggttgaccttgccaagccctccaggtgatgcgcggccatgccgggtgaggagaactgaggctgagatcgccacactgagggctccctaaagccgaggtggaggaagaggagggtagaggaggagggcggtattgctgggaaccgccccctccctgccctgctccctgctgccccacccgagccct

SEQ ID NO: 15 - DNA, 3000 bp, Homo sapiens
gaatacattaaagtaggggcaacccttgagcccagacttctgccatgtgaagaccctttgaaaatcctgacaaacacaggtactgcgtaagtggtcagctaattaaagaggggaggtggagctgtcctttgtgtatccaataagtacccattatctcatttgagcatgaaaagaggccactgttattactttcaagaaggaaagtaagcaggatagctcatatttttagaaccattcctcaccaaatggaataattccggtgaaaagtgggagtgaggaagaaagaaaaaaaaaacttctaatcataatgtttgggaataagaaaggaagaagaaactcacgtcaaagccgactttctcctgcagctgtaaaataaactcttaagacccttcctgctgaaactctggagaggaaaactggagtggcgggtgggctttgcctgcagctcaactctccctcgcggcgcgggcgcggctgggttcagcacctcggaaagcgcccctcgcggcgccccgggattacgcatgctccttggggcccgccgccttggccgtgcaagtgccaccgtaactggtgagagccgctggcaacccacccggagttgacaaccgcggagagacgcagacacccactgacctccaggaagctgagcgtggtggatggaactctacgatctctttctctccaaggacggaaacctcatccaagcagtcccagaggaaacggataaaggtatttgaaagggagcgagcggccccaaatcgcacaattgagcggctgggggagttatgcgccagtgccccagtgaccgcgggacacggagaggggaagtctgcgttgtacataaggacctagggactccgagcttggcctgagaacccttggacgccgagtgcttgccttacgggctgcactcctcaactctgctccaaagcagccgctgagctcaactcctgcgtccagggcgttcgctgcgcgccaggacgcgcttagtacccagttcctgggctctctcttcagtagctgctttgaaagctcccacgcacgtcccgcaggctagcctggcaacaaaactggggtaaaccgtgttatcttaggtcttgtcccccagaacatgacctagaggtacctgcgcatgcagatggccgatgcagccacgatagccaccatgaataaggcagcaggcggggacaagctagcagaactcttcagtctggtcccggaccttctggaggcggccaacacgagtggtaacgcgtcgctgcagcttccggacttgtggtgggagctggggctggagttgccggacggcgcgccgccaggacatcccccgggcagcggcggggcagagagcgcggacacagaggcccgggtgcggattctcatcagcgtggtgtactgggtggtgtgcgccctggggttggcgggcaacctgctggttctctacctgatgaagagcatgcagggctggcgcaagtcctctatcaacctcttcgtcaccaacctggcgctgacggactttcagtttgtgctcaccctgcccttctgggcggtggagaacgctcttgacttcaaatggcccttcggcaaggccatgtgtaagatcgtgtccatggtgacgtccatgaacatgtacgccagcgtgttcttcctcactgccatgagtgtgacgcgctaccattcggtggcctcggctctgaagagccaccggacccgaggacacggccggggcgactgctgcggccggagcctgggggacagctgctgcttctcggccaaggcgctgtgtgtgtggatctgggctttggccgcgctggcctcgctgcccagtgccattttctccaccacggtcaaggtgatgggcgaggagctgtgcctggtgcgtttcccggacaagttgctgggccgcgacaggcagttctggctgggcctctaccactcgcagaaggtgctgctgggcttcgtgctgccgctgggcatcattatcttgtgctacctgctgctggtgcgcttcatcgccgaccgccgcgcggcggggaccaaaggaggggccgcggtagccggaggacgcccgaccggagccagcgcccggagactgtcgaaggtcaccaaatcagtgaccatcgttgtcctgtccttcttcctgtgttggctgcccaaccaggcgctcaccacctggagcatcctcatcaagttcaacgcggtgcccttcagccaggagtatttcctgtgccaggtatacgcgttccctgtgagcgtgtgcctagcgcactccaacagctgcctcaaccccgtcctctactgcctcgtgcgccgcgagttccgcaaggcgctcaagagcctgctgtggcgcatcgcgtctccttcgatcaccagcatgcgccccttcaccgccactaccaagccggagcacgaggatcaggggctgcaggccccggcgccgccccacgcggccgcggagccggacctgctctactacccacctggcgtcgtggtctacagcggggggcgctacgacctgctgcccagcagctctgcctactgacgcaggcctcaggcccagggcgcgccgtcggggcaaggtggccttccccgggcggtaaagaggtgaaaggatgaaggagggctggggggggccccatttaagaagtaggtgggaggaggatgggcagagcatggaggaggagcctgtggataggccgaggaccttctctggagaggagatgcttcgaaatcaggtggagagaggaaattggcaaagggatagagacgagccccacgggccagacagccaacctccgctccgcaccccacagcctctccttactcttcccacgctgagtagtgtgggggcgcccagaagcgaagacaagcagcaaaaatgtagagaaattggcacggggagcggggcttagccaaatgatgcacagacaattgtgcccgtttattccagcgacttctgcggagagggcagccgtcggcacaaacactcctttgcgt

SEQ ID NO: 16 - DNA, 2200 bp, Homo sapiens
gtcccccgattccctcacccatcatataacgtgtgtatttattatgtttcccgtttcctctgtctccgccagcagaatgtaaactccatgaggtcaggaatctccgagttatgttgcgccagtgtaatccaagagcccggaacagtgcctggcacacagcgggcatatggaagaacaaatgtgtgaaggtgtgaatgaatgaataattgaaagaataaatagtagttctcagcctcacagaacacgggtcacaacctcaaatgacctgctaccctgcccataaataacagagatgcaggagtaagtgctgggctgtgacctgtcaacatgctaagccgctcaaacaaaactgcccaacagcccgctggccgcctatttgcagcactgggccctgagccgcacattcccatttcgttgataaagaaactgaccagatagtttaagtggcctgctgcggaagacagagctggtgctgcaccggtcgctgcttccccagtccttttttggcctcctttctgacgcgacgcagaccccagttctggagagtctgtcactcgctccccgtggtgggagatcagaggcctggtgtccttgggagcggcgagcggtgctcggcgcaggatagaaagggagtgcgcgcccgagtcccccagatccctgggaacccgcgccaccctcccgcccctgcccatccccggccgcgctgtcagtctccattagcgctaacaggctccagacggagcgggccgggcgctgggttaatgcaatcggcgcgttacctggggcgcaggctacattaccagcccggcccccgccaggcacggccagaaccagtcagcccgcgccctgccggccgccccgcgcctccagctcttccccggccccgcccgaacgccacacggcggagcccagccccagcccgcgccctagagcctgccaaggcgccgccggtcgggggccggcagggcgcaaggcaccagggatcccctcgccgccggacacgtgagtgcgccctgagcgcgggacagggctaggtctgcctgggaggcccgggccgagacgcgccagcagagggctagcgagtttgtagtgcagtgacgttaagtgtccgagaaggctcctgtggctgttgaagtgtcgcggacctgagctggggagggggtcggcacgctgccctcagcctcggtgagttcaatcccagccatttggggcaggcgagagtgggtgaacgaggaaaagtgctgcagggtcttcagccgcccccagagggctgtcagaagtctccaactcttgagttccggcgtgccccaacctctgtttccaaatttttccagcggacgcgcgctcttttctgggaaccctgcgtccgctcagcgcgcgctcatcccagtgtctaaggcgctcccgggtggtcttgggagttgcaagtagggaggaacggccgggtaaccacctcttttccctttatccaagcagagcctcggcgtgcccccaggaccggtaaagttcctctcgccagccgcatccatgcttctggcgcggatgaacccgcaggtgcagcccgagaacaacggggcggacacgggtccagagcagccccttcgggcgcgcaaaactgcggagctgctggtggtgaaggagcgcaacggcgtccagtgcctgctggcgccccgcgacggcgacgcgcagccccgggagacctggggcaagaagatcgacttcctgctgtccgtagtcggcttcgcagtggacctggccaacgtgtggcgcttcccctacctctgctacaagaacggcggcggtgagcgtggggtcgggctgggaatttgaatctgggaggtccactgtctgcagcggtggctgggacaggagctggaatacacacggaagggaggcgaggagacaggggcaaatctggggcgcagaaagaactggacagggctaacgggaaaaaaaaaagattggagtcctctggaaggtcattttcccaggctctttgcagagtacctcgagctcattccagcggaagtgtcaggattgggcaccctggaagcaaaacagcagaagagtgaaatcgagtcatgaccctaaagtcatggtaggggtatggatggaaaggacagaatctggggtgccaggttgggtgggggagcctgaccttttgatggtctgctggaagggaggtggagattccaagagc

SEQ ID NO: 17 - DNA, 98 bp, Artificial Sequence
  Designed methylated oligonucleotide for experiment
gttggccactgcggagtcgngcngggtggcnggccgcacctacagngccgngngngcggtgagcctggccgtgtgggggatcgtcacactcgtcgtgc

SEQ ID NO: 18 - DNA, 98 bp, Artificial Sequence
  Designed unmethylated oligonucleotide for experiment
gttggccactgcggagtcgcgccgggtggccggccgcacctacagcgccgcgcgcgcggtgagcctggccgtgtgggggatcgtcacactcgtcgtgc

SEQ ID NO: 19 - DNA, 20 bp, Artificial Sequence
  Designed biotinated oligonucleotide for imobilization on support material
gcacgacgagtgtgacgatc

SEQ ID NO: 20 - DNA, 14 bp, Artificial Sequence
  Designed oligonucleotide for experiment
gccacccggcgcga

SEQ ID NO: 21 - DNA, 19 bp, Artificial Sequence
  Designed oligonucleotide for experiment
gttggccactgcggagtcg

SEQ ID NO: 22 - DNA, 20 bp, Artificial Sequence
  Designed oligonucleotide primer for PCR
gcacgacgagtgtgacgatc

SEQ ID NO: 23 - DNA, 98 bp, Artificial Sequence; identical to SEQ ID NO: 18
  Designed oligonucleotide consist of objective DNA domain (GPR7-2079-2176, methylated cytosin is also shown as C)

SEQ ID NO: 24 - DNA, 96 bp, Homo sapiens
cacctggaaaatcgggtcactcccacccgaatattgcgcttttcagaccggcttaagaaacggcgcaccacgagactatatcccacacctggctcg

SEQ ID NO: 25 - DNA, 473 bp, Homo sapiens
gagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttctgagtcaaagaaaggggtgacggtcgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcag

SEQ ID NO: 26 - DNA, 21 bp, Artificial Sequence
  Designed oligonucleotide primer for PCR
gagccaagatggccgaatagg

SEQ ID NO: 27 - DNA, 23 bp, Artificial Sequence
  Designed oligonucleotide primer for PCR
ctgctttgtttacctaagcaagc

SEQ ID NO: 28 - DNA, 21 bp, Artificial Sequence
  Designed oligonucleotide primer for Real Time PCR
cacctggaaaatcgggtcact

SEQ ID NO: 29 - DNA, 20 bp, Artificial Sequence
  Designed oligonucleotide primer for Real Time PCR
cgagccaggtgtgggatata

SEQ ID NO: 30 - DNA, 27 bp, Artificial Sequence
  Designed oligonucleotide probe for Real Time PCR
cgaatattgcgcttttcagaccggctt

(19) United States
(12) Patent Application Publication
(10) Pub. No.: US 2010/0009376 A1
(43) Pub. Date: Jan. 14, 2010

(54) METHOD FOR MEASURING DNA METHYLATION

(75) Inventors: Yoshitaka Tomigahara, Osaka (JP); Hirokazu Tarui, Osaka (JP)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: SUMITOMO CHEMICAL COMPANY, LIMITED, Chuo-ku, Tokyo (JP)

(21) Appl. No.: 12/525,089

(22) PCT Filed: Jan. 31, 2008
(86) PCT No.: PCT/JP2008/051995

§ 371 Date: Jul. 30, 2009

(30) Foreign Application Priority Data
Jan. 31, 2007 (JP) 2007-021171

(51) Int. Cl. C12Q 1/68

(52) U.S. Cl. 435/6

(57) ABSTRACT

The present invention relates to a method of measuring the content of methylated DNA in a DNA region of interest in a genomic DNA contained in a biological specimen, and so on.

### TECHNICAL FIELD

The present invention relates to a method of measuring the content of methylated DNA in a DNA region of interest in a genomic DNA contained in a biological specimen, and so on.

### BACKGROUND ART

As a method for evaluating the methylation state of DNA in an objective DNA region in a genomic DNA contained in a biological specimen, for example, there is known a method of measuring the content of methylated DNA in an objective DNA region in a genomic DNA (see, for example, Nucleic Acids Res., 1994, Aug. 11; 22(15): 2990-7, and Proc. Natl. Acad. Sci. U.S.A., 1997, Mar. 18; 94(6): 2284-9 for reference). In such a measuring method, first, it is necessary to extract DNA containing the objective DNA region from a DNA sample derived from a genomic DNA, and the extracting operation is complicated.

As a method of measuring the content of methylated DNA in an objective region of extracted DNA, for example, (1) a method of amplifying an objective region by subjecting the DNA to a chain reaction for DNA synthesis by DNA polymerase after modification of the DNA with a sulfite or the like (Polymerase Chain Reaction; hereinafter also referred to as PCR), and (2) a method of amplifying an objective region by subjecting the DNA to PCR after digestion of the DNA using a methylation sensitive restriction enzyme are known. Both of these methods require time and labor for DNA modification for detection of methylation, subsequent purification of the product, preparation of a reaction system for PCR, and checking of DNA amplification.

### DISCLOSURE OF THE INVENTION

It is an object of the present invention to provide a method of measuring the content of methylated DNA in an objective DNA region in a genomic DNA contained in a biological specimen in a simple and convenient manner.

That is, the present invention provides:

1. a method of measuring the content of methylated DNA in an objective DNA region in a genomic DNA contained in a biological specimen, comprising:
either one of the following combined steps:
(i) combined step (i) comprising:
(1) First step having:

First (A) step of mixing single-stranded DNA (plus strand) containing an objective DNA region with a masking oligonucleotide having a nucleotide sequence complementary to the nucleotide sequence of a recognition site of a methylation sensitive restriction enzyme, thereby generating from a DNA sample derived from genomic DNA contained in a biological specimen single-stranded DNA in which the recognition site of the methylation sensitive restriction enzyme is protected, and

First (B) step of causing base-pairing between the single-stranded DNA (plus strand) containing the objective DNA region protected and generated in First (A) step and a single-stranded immobilized oligonucleotide having a nucleotide sequence complementary to a part (provided that, not containing the objective DNA region) of the 3′-end of the single-stranded DNA, thereby selecting the protected and generated single-stranded DNA, and

(2) Second step of digesting the single-stranded DNA selected in First step with one or more kinds of methylation-sensitive restriction enzyme, and then removing a generated free digest (single-stranded DNA containing one or more unmethylated CpGs in the recognition site of the methylation sensitive restriction enzyme, the site being protected by the masking oligonucleotide); or,
(ii) combined step (ii) comprising:
(1) First step of causing base-pairing between a single-stranded DNA (plus strand) containing an objective DNA region and a single-stranded immobilized oligonucleotide having a nucleotide sequence complementary to a part (provided that, not containing the objective DNA region) of the 3′-end of the single-stranded DNA, thereby selecting the single-stranded DNA from a DNA sample derived from genomic DNA contained in a biological specimen, and
(2) Second step having:

Second (A) step of mixing the single-stranded DNA selected in First step, with a masking oligonucleotide having a nucleotide sequence complementary to the nucleotide sequence of a recognition site of a methylation-sensitive restriction enzyme, thereby generating a single-stranded DNA in which the recognition site of the methylation-sensitive restriction enzyme is protected, and

Second (B) step of digesting the single-stranded DNA protected and generated in Second (A) step with one or more kinds of methylation-sensitive restriction enzyme, and removing a generated free digest (single-stranded DNA containing one or more unmethylated CpGs in the recognition site of the methylation-sensitive restriction enzyme, the site being protected by the masking oligonucleotide); and

(3) Third step comprising as a pre step of each of the following regular steps:

a step (First pre step) of temporarily separating a single-stranded DNA which is an undigested substance obtained in Second step (single-stranded DNA not containing unmethylated CpG in the recognition site of the methylation sensitive restriction enzyme, the site being protected by the masking oligonucleotide) from both of the single-stranded immobilized oligonucleotide and the masking oligonucleotide, and

a step (Second pre step) having

a step (Second (A) pre step) of causing base-pairing between the generated single-stranded DNA (plus strand) and a single-stranded oligonucleotide, thereby selecting the generated single-stranded DNA and forming DNA in which the selected single-stranded DNA and the single-stranded oligonucleotide are base-paired, and

a step (Second (B) pre step) of making the DNA formed in the step (Second (A) pre step) into double-stranded DNA in which the selected single-stranded DNA has been extended by allowing one extension of a primer by using the selected single-stranded DNA as a template and the single-stranded oligonucleotide as a primer, and

a step (Third pre step) of temporarily separating the double-stranded DNA extensionally-formed in Second pre step (extensionally-formed double-stranded DNA not containing an unmethylated CpG pair in the recognition site of the methylation-sensitive restriction enzyme, the site being protected by the masking oligonucleotide) into a single-stranded DNA (plus strand) and a single-stranded DNA (minus strand), and as regular steps:

(a) Regular step A having Step A1 of selecting the single-stranded DNA by causing base-pairing between the generated single-stranded DNA (plus strand) and the single-stranded immobilized oligonucleotide (minus strand), and Step A2 of extensionally-forming double-stranded DNA from the single-stranded DNA by causing one extension of a primer by using single-stranded DNA selected in Step A1 as a template and the single-stranded immobilized oligonucleotide as the primer, and

(b) Regular step B of extensionally-forming double-stranded DNA from the single-stranded DNA by causing one extension of an extension primer by using the generated single-stranded DNA (minus strand) as a template, and the extension primer (reverse primer) having a nucleotide sequence (plus strand) complementary to a partial nucleotide sequence (minus strand) of nucleotide sequence possessed by the single-stranded DNA (minus strand), wherein the partial nucleotide sequence (minus strand) is positioned on the 3′-end side than the 3′-end of the nucleotide sequence (minus strand) complementary to the nucleotide sequence (plus strand) of the objective DNA region as an extension primer,

wherein the methylated DNA in the objective DNA region is amplified to a detectable level by repeating each regular step after temporarily separating the extensionally-formed double-stranded DNA obtained in each regular step into a single-stranded state, and amount of the amplified DNA is quantified (hereinafter, also referred to as present measuring method);

2. the method according to the item 1, wherein in First step, base pairing is conducted in a reaction system containing a divalent cation when the single-stranded DNA containing an objective DNA region (plus strand) and the single-stranded immobilized oligonucleotide having a nucleotide sequence complementary to a part (provided that, not containing the objective DNA region) of the 3′-end of the single-stranded DNA are base-paired;
3. the method according to the item 2, wherein the divalent cation is a magnesium ion;
4. the method according to any one of the items 1 to 3, further comprising prior to First pre step in Third step:

a step (Additional pre step) of adding into the reaction system a single-stranded oligonucleotide (minus strand) in a free state having a nucleotide sequence complementary to a part of the 3′-end of the single-stranded DNA (plus strand) containing the objective DNA region, and further comprising the following one step as a respective regular step of Third step as described in the item 1:

(c) Regular step C having:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by allowing one extension of a primer by using the single-stranded DNA selected in Step C1 as a template and the single-stranded oligonucleotide (minus strand) as a primer;

5. the method according to any one of the items 1 to 3, further comprising after First pre step in Third step:

a step (Additional pre step) of adding into the reaction system a single-stranded oligonucleotide (minus strand) in a free state having a nucleotide sequence complementary to a part of the 3′-end of the single-stranded DNA (plus strand) containing the objective DNA region, and further comprising the following one step as a respective regular step of Third step as described in the item 1:

(c) Regular step C having:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by allowing one extension of a primer by using the single-stranded DNA selected in Step C1 as a template and the single-stranded oligonucleotide (minus strand) as a primer;

6. the method according to any one of the items 1 to 3, further comprising prior to Third pre step in Third step:

a step (Additional pre step) of adding into the reaction system a single-stranded oligonucleotide (minus strand) in a free state having a nucleotide sequence complementary to a part of the 3′-end of the single-stranded DNA (plus strand) containing the objective DNA region, and further comprising the following one step as a respective regular step of Third step as described in the item 1:

(c) Regular step C having:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by allowing one extension of a primer by using the single-stranded DNA selected in Step C1 as a template and the single-stranded oligonucleotide (minus strand) as a primer;

7. the method according to any one of the items 1 to 3, further comprising after Third pre step in Third step:

a step (Additional pre step) of adding into the reaction system a single-stranded oligonucleotide (minus strand) in a free state having a nucleotide sequence complementary to a part of the 3′-end of the single-stranded DNA (plus strand) containing the objective DNA region, and further comprising the following one step as a respective regular step of Third step as described in the item 1:

(c) Regular step C having:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by allowing one extension of a primer by using the single-stranded DNA selected in Step C1 as a template and the single-stranded oligonucleotide (minus strand) as a primer;

8. the method of measuring a methylation rate (hereinafter, also referred to as present methylation rate measuring method) further comprising the following two steps as steps of the method according to any one of the items 1 to 7:
(4) Fourth step of amplifying DNA (total amount of methylated DNA and unmethylated DNA) of the objective DNA region to a detectable level by conducting Third step in the method according to any of the items 1 to 7 after conducting First step in the method according to any one of the items 1 to 7 without conducting Second step of combined step (i) or Second (B) step of combined step (ii) in the method according to any one of the items 1 to 7, and quantifying the amplified DNA; and
(5) Fifth step of calculating a rate of methylated DNA in the objective DNA region based on a difference obtained by comparing the DNA amount quantified by Third step according to any one of the items 1 to 7, and the DNA amount quantified in Fourth step;
9. the method according to any one of the items 1 to 8, wherein the biological specimen is mammalian serum or plasma;
10. the method according to any one of the items 1 to 8, wherein the biological specimen is mammalian blood or bodily secretion;
11. the method according to any one of the items 1 to 8, wherein the biological specimen is a cell lysate or a tissue lysate;
12. the method according to any one of the items 1 to 11, wherein the DNA sample derived from a genomic DNA contained in a biological specimen is a DNA sample digested in advance with a restriction enzyme whose recognition cleavage site excludes the objective DNA region possessed by the genomic DNA;
13. the method according to any one of the items 1 to 12, wherein the DNA sample derived from a genomic DNA contained in a biological specimen is a DNA sample digested with one or more kinds of methylation sensitive restriction enzyme;
14. the method according to any one of the items 1 to 13, wherein the DNA sample derived from a genomic DNA contained in a biological specimen is a DNA sample purified in advance;
15. the method according to any one of the items 1 to 14, wherein the one or more kinds of methylation sensitive restriction enzyme is a restriction enzyme having its recognition cleavage site in the objective DNA region possessed by a genomic DNA contained in the biological specimen; and
16. the method according to any one of the items 1 to 15, wherein the one or more kinds of methylation sensitive restriction enzyme is HpaII or HhaI which is a methylation sensitive restriction enzyme; and so on.

### BRIEF DESCRIPTION OF THE DRAWINGS

FIG. 1 shows results of 1.5% agarose gel electrophoresis of amplified products which are obtained by conducting a treatment of either “A (no treatment)”, “B (HpaII treatment)” or “C (addition of masking oligonucleotide+HpaII treatment)” on the sample prepared in Example 1, and amplifying methylated DNA in the region having the nucleotide sequence of SEQ ID NO:23 by PCR. In the drawing, results of a sample subjected to “A” treatment of methylated oligonucleotide GPR7-2079-2176/98mer-M(7)(M) in which the HpaII recognition sequence is methylated, a sample subjected to “B” treatment of methylated oligonucleotide GPR7-2079-2176/98mer-M(7)(M) in which the HpaII recognition sequence is methylated, a sample subjected to “C” treatment of methylated oligonucleotide GPR7-2079-2176/98mer-M(7)(M) in which the HpaII recognition sequence is methylated, a sample subjected to “A” treatment of unmethylated oligonucleotide GPR7-2079-2176/98mer-UM(U) in which the HpaII recognition sequence is not methylated, a sample subjected to “B” treatment of unmethylated oligonucleotide GPR7-2079-2176/98mer-UM(U) in which the HpaII recognition sequence is not methylated, and a sample subjected to “C” treatment of unmethylated oligonucleotide GPR7-2079-2176/98mer-UM(U) in which the HpaII recognition sequence is not methylated are shown from the leftmost lane.

FIG. 2 shows results of 1.5% agarose gel electrophoresis of amplified products which are obtained by conducting a treatment of either “A (no treatment)”, “B (HpaII treatment)” or “C (addition of masking oligonucleotide+HpaII treatment)” on the sample prepared in Example 2, and amplifying methylated DNA in the region having the nucleotide sequence of SEQ ID NO:23 by PCR. In the drawing, results of a sample subjected to “A” treatment of methylated oligonucleotide GPR7-2079-2176/98mer-M(7)(M) in which the HpaII recognition sequence is methylated, a sample subjected to “B” treatment of methylated oligonucleotide GPR7-2079-2176/98mer-M(7)(M) in which the HpaII recognition sequence is methylated, a sample subjected to “C” treatment of methylated oligonucleotide GPR7-2079-2176/98mer-M(7)(M) in which the HpaII recognition sequence is methylated, a sample subjected to “A” treatment of unmethylated oligonucleotide GPR7-2079-2176/98mer-UM(U) in which the HpaII recognition sequence is not methylated, a sample subjected to “B” treatment of unmethylated oligonucleotide GPR7-2079-2176/98mer-UM(U) in which the HpaII recognition sequence is not methylated, and a sample subjected to “C” treatment of unmethylated oligonucleotide GPR7-2079-2176/98mer-UM(U) in which the HpaII recognition sequence is not methylated are shown from the leftmost lane.

FIG. 3 shows results of the amount of methylated DNA in a region having the nucleotide sequence of SEQ ID NO: 24 measured by real-time PCR, for the sample “(I)” prepared in Example 3, subjected to either “A (no treatment)”, “B (HpaII treatment)”, “C (HhaI treatment)” or “D (co-treated with HpaII and HhaI)”. The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C and Group D are indicated.

FIG. 4 shows results of the amount of methylated DNA in a region having the nucleotide sequence of SEQ ID NO.: 24 measured by real-time PCR, for the sample “(II)” prepared in Example 3, subjected to either “A (no treatment)”, “B (HpaII treatment)”, “C (HhaI treatment)” or “D (co-treated with HpaII and HhaI)”. The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C and Group D are indicated.

FIG. 5 shows results of the amount of methylated DNA in a region having the nucleotide sequence of SEQ ID NO.: 24 measured by real-time PCR, for the sample “(III)” prepared in Example 3, subjected to either “A (no treatment)”, “B (HpaII treatment)”, “C (HhaI treatment)” or “D (co-treated with HpaII and HhaI)”.

The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C and Group D are indicated.

FIG. 6 shows results of the amount of methylated DNA in a region having the nucleotide sequence of SEQ ID NO.: 24 measured by real-time PCR, for the sample “(IV)” prepared in Example 3, subjected to either “A (no treatment)”, “B (HpaII treatment)”, “C (HhaI treatment)” or “D (co-treated with HpaII and HhaI)”. The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C and Group D are indicated.

FIG. 7 results of amount of the methylated DNA in a region having the nucleotide sequence of SEQ ID NO.: 24 measured by real-time PCR, for the sample “(V)” prepared in Example 3, subjected to either “A (no treatment)”, “B (HpaII treatment)”, “C (HhaI treatment)” or “D (co-treated with HpaII and HhaI)”. The vertical axis in the drawing represents the relative value when the amount of DNA in the sample subjected to “A” treatment is assumed to be 1 (average value of three measurements±standard deviation). As theoretical values, calculated values (methylation rate) expected in Group B, Group C and Group D are indicated.

### BEST MODE FOR CARRYING OUT THE INVENTION

As the “biological specimen” in the present invention, for example, a cell lysate, a tissue lysate (here the term “tissue” is used in a broad sense including blood, lymph node and so on) or biological samples including bodily sections such as plasma, serum and lymph, bodily secretions (urine, milk and so on) and the like and a genomic DNA obtained by extracting these biological samples, in mammals can be recited. As a biological specimen, for example, samples derived from microorganisms, viruses and the like can be recited, and in such a case, “a genomic DNA” in the present measuring method also means genomic DNA of microorganisms, viruses and the like.

When the specimen derived from a mammal is blood, use of the present measuring method in a regular health check or a simple examination is expected.

For obtaining a genomic DNA from a specimen derived from a mammal, for example, DNA may be extracted using a commercially available DNA extraction kit.

When blood is used as a specimen, plasma or serum is prepared from blood in accordance with a commonly used method, and using the prepared plasma or serum as a specimen, free DNA (including DNA derived from cancer cells such as gastric cancer cells) contained in the specimen is analyzed. This enables analysis of DNA derived from cancer cells such as gastric cancer cells while avoiding DNA derived from hemocytes, and improves the sensitivity of detection of cancer cells such as gastric cancer cells and a tissue containing the same.

Usually, a gene (a genomic DNA) consists of four kinds of bases. In these bases, such a phenomenon is known that only cytosine is methylated, and such methylation modification of DNA is limited to cytosine in the nucleotide sequence represented by 5′-CG-3′ (C represents cytosine, and G represents guanine. Hereinafter, the nucleotide sequence is also referred to as “CpG”) The site to be methylated in cytosine is its position 5. In DNA replication prior to cell division, only cytosine in “CpG” of a template chain is methylated immediately after replication, however, cytosine in “CpG” of a newly-generated strand is immediately methylated by the action of methyltransferase. Therefore, the methylation state of DNA will be passed to new two sets of DNA even after DNA replication. The term “methylated DNA” in the present invention means DNA occurring by such methylation modification.

The term “CpG pair” in the present invention means double-stranded oligonucleotide in which the nucleotide sequence represented by CpG and a CpG that is complement with this are base-paired.

The term “objective DNA region” (hereinafter, also referred to as an “objective region”) used in the present invention means a DNA region for which presence or absence of methylation of cytosine included in the region is to be examined, and has a recognition site of one or more kinds of methylation sensitive restriction enzyme. A DNA region containing at least one cytosine in the nucleotide sequence represented by CpG which is present in a nucleotide sequence of a promoter region, an untranslated region, or a translated region (coding region) of a useful protein gene such as Lysyl oxidase, HRAS-like suppressor, bA305P22.2.1, Gamma filamin, HAND1, Homologue of RIKEN 2210016F16, FLJ32130, PPARG angiopoietin-related protein, Thrombomodulin, p53-responsive gene 2, Fibrillin2, Neurofilament3, disintegrin and metalloproteinase domain 23, G protein-coupled receptor 7, G-protein coupled somatostatin and angiotensin-like peptide receptor, Solute carrier family 6 neurotransmitter transporter noradrenalin member 2 and so on can be recited.

To be more specific, when the useful protein gene is a Lysyl oxidase gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a Lysyl oxidase gene derived from human, and a promoter region located 5′ upstream of the same can be recited, and more concretely, the nucleotide sequence of SEQ ID NO: 1 (corresponding to the nucleotide sequence represented by base No. 16001 to 18661 in the nucleotide sequence described in Genbank Accession No. AF270645) can be recited. In the nucleotide sequence of SEQ ID NO: 1, ATG codon encoding methionine at amino terminal of Lysyl oxidase protein derived from human is represented in base No. 2031 to 2033, and a nucleotide sequence of the above exon 1 is represented in base No. 1957 to 2661. Cytosine in the nucleotide sequence represented by CpG which is present in the nucleotide sequence of SEQ ID NO: 1, in particular, cytosine in CpG which is present in a region where CpGs are densely present in the nucleotide sequence of SEQ ID NO: 1 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1539, 1560, 1574, 1600, 1623, 1635, 1644, 1654, 1661, 1682, 1686, 1696, 1717, 1767, 1774, 1783, 1785, 1787, 1795 and so on in the nucleotide sequence of SEQ ID NO: 1 can be recited.

To be more specific, when the useful protein gene is a HRAS-like suppressor gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a HRAS-like suppressor gene derived from human, and a promoter region located 5′ upstream of the same can be recited, and more concretely, the nucleotide sequence of SEQ ID NO: 2 (corresponding to the nucleotide sequence represented by base No. 172001 to 173953 in the nucleotide sequence described in Genbank Accession No. AC068162) can be recited. In the nucleotide sequence of SEQ ID NO: 2, the nucleotide sequence of exon 1 of a HRAS-like suppressor gene derived from human is represented in base No. 1743 to 1953. Cytosine in the nucleotide sequence represented by CpG which is present in the nucleotide sequence of SEQ ID NO: 2, in particular, cytosine in CpG which is present in a region where CpGs are densely present in the nucleotide sequence of SEQ ID NO: 2 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1316, 1341, 1357, 1359, 1362, 1374, 1390, 1399, 1405, 1409, 1414, 1416, 1422, 1428, 1434, 1449, 1451, 1454, 1463, 1469, 1477, 1479, 1483, 1488, 1492, 1494, 1496, 1498, 1504, 1510, 1513, 1518, 1520 and so on in the nucleotide sequence of SEQ ID NO: 2 can be recited.

To be more specific, when the useful protein gene is a bA305P22.2.1 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a bA305P22.2.1 gene derived from human, and a promoter region located 5′ upstream of the same can be recited, and more concretely, the nucleotide sequence of SEQ ID NO: 3 (corresponding to the nucleotide sequence represented by base No. 13001 to 13889 in the nucleotide sequence described in Genbank Accession No. AL121673) can be recited. In the nucleotide sequence of SEQ ID NO: 3, ATG codon encoding methionine at amino terminal of bA305P22.2.1 protein derived from human is represented in base No. 849 to 851, and a nucleotide sequence of the above exon 1 is represented in base No. 663 to 889. Cytosine in the nucleotide sequence represented by CpG which is present in the nucleotide sequence of SEQ ID NO: 3, in particular, cytosine in CpG which is present in a region where CpGs are densely present in the nucleotide sequence of SEQ ID NO: 3 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 329, 335, 337, 351, 363, 373, 405, 424, 427, 446, 465, 472, 486 and so on in the nucleotide sequence of SEQ ID NO: 3 can be recited.

To be more specific, when the useful protein gene is a Gamma filamin gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a Gamma filamin gene derived from human, and a promoter region located 5′ upstream of the same can be recited, and more concretely, the nucleotide sequence of SEQ ID NO: 4 (corresponding to a complementary sequence to the nucleotide sequence represented by base No. 63528 to 64390 in the nucleotide sequence described in Genbank Accession No. AC074373) can be recited. In the nucleotide sequence of SEQ ID NO: 4, ATG codon encoding methionine at amino terminal of Gamma filamin protein derived from human is represented in base No. 572 to 574, and a nucleotide sequence of the above exon 1 is represented in base No. 463 to 863. Cytosine in the nucleotide sequence represented by CpG which is present in the nucleotide sequence of SEQ ID NO: 4, in particular, cytosine in CpG which is present in a region where CpGs are densely present in the nucleotide sequence of SEQ ID NO: 4 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 329, 333, 337, 350, 353, 360, 363, 370, 379, 382, 384, 409, 414, 419, 426, 432, 434, 445, 449, 459, 472, 474, 486, 490, 503, 505 and so on in the nucleotide sequence of SEQ ID NO: 4 can be recited.

To be more specific, when the useful protein gene is a HAND1 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a HAND1 gene derived from human, and a promoter region located 5′ upstream of the same can be recited, and more concretely, the nucleotide sequence of SEQ ID NO: 5 (corresponding to a complementary sequence to the nucleotide sequence represented by base No. 24303 to 26500 in the nucleotide sequence described in Genbank Accession No. AC026688) can be recited. In the nucleotide sequence of SEQ ID NO: 5, ATG codon encoding methionine at amino terminal of HAND1 protein derived from human is represented in base No. 1656 to 1658, and a nucleotide sequence of the above exon 1 is represented in base No. 1400 to 2198. Cytosine in the nucleotide sequence represented by CpG which is present in the nucleotide sequence of SEQ ID NO: 5, in particular, cytosine in CpG which is present in a region where CpGs are densely present in the nucleotide sequence of SEQ ID NO: 5 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1153, 1160, 1178, 1187, 1193, 1218, 1232, 1266, 1272, 1292, 1305, 1307, 1316, 1356, 1377, 1399, 1401, 1422, 1434 and so on in the nucleotide sequence of SEQ ID NO: 5 can be recited.

To be more specific, when the useful protein gene is a Homologue of RIKEN 2210016F16 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a Homologue of RIKEN 2210016F16 gene derived from human, and a promoter region located 5′ upstream of the same can be recited, and more concretely, the nucleotide sequence of SEQ ID NO: 6 (corresponding to a complementary nucleotide sequence to the nucleotide sequence represented by base No. 157056 to 159000 in the nucleotide sequence described in Genbank Accession No. AL354733) can be recited. In the nucleotide sequence of SEQ ID NO: 6, a nucleotide sequence of exon 1 of a Homologue of a RIKEN 2210016F16 gene derived from human is represented in base No. 1392 to 1945. Cytosine in the nucleotide sequence represented by CpG which is present in the nucleotide sequence of SEQ ID NO: 6, in particular, cytosine in CpG which is present in a region where CpGs are densely present in the nucleotide sequence of SEQ ID NO: 6 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1172, 1175, 1180, 1183, 1189, 1204, 1209, 1267, 1271, 1278, 1281, 1313, 1319, 1332, 1334, 1338, 1346, 1352, 1358, 1366, 1378, 1392, 1402, 1433, 1436, 1438 and so on in the nucleotide sequence of SEQ ID NO: 6 can be recited.

To be more specific, when the useful protein gene is a FLJ32130 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a FLJ32130 gene derived from human, and a promoter region located 5′ upstream of the same can be recited, and more concretely, the nucleotide sequence of SEQ ID NO: 7 (corresponding to a complementary nucleotide sequence to the nucleotide sequence represented by base No. 1 to 2379 in the nucleotide sequence described in Genbank Accession No. AC002310) can be recited. In the nucleotide sequence of SEQ ID NO: 7, ATG codon encoding methionine at amino terminal of FLJ32130 protein derived from human is represented in base No. 2136 to 2138, and a nucleotide sequence assumed to be the above exon 1 is represented in base No. 2136 to 2379. Cytosine in the nucleotide sequence represented by CpG which is present in the nucleotide sequence of SEQ ID NO: 7, in particular, cytosine in CpG which is present in a region where CpGs are densely present in the nucleotide sequence of SEQ ID NO: 7 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1714, 1716, 1749, 1753, 1762, 1795, 1814, 1894, 1911, 1915, 1925, 1940, 1955, 1968 and so on in the nucleotide sequence of SEQ ID NO: 7 can be recited.

To be more specific, when the useful protein gene is a PPARG angiopoietin-related protein gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a PPARG angiopoietin-related protein gene derived from human, and a promoter region located 5′ upstream of the same can be recited, and more concretely, the nucleotide sequence of SEQ ID NO: 8 can be recited. In the nucleotide sequence of SEQ ID NO: 8, ATG codon encoding methionine at amino terminal of PPARG angiopoietin-related protein derived from human is represented in base No. 717 to 719, and a nucleotide sequence of the 5′-end part of the above exon 1 is represented in base No. 1957 to 2661. Cytosine in the nucleotide sequence represented by CpG which is present in the nucleotide sequence of SEQ ID NO: 8, in particular, cytosine in CpG which is present in a region where CpGs are densely present in the nucleotide sequence of SEQ ID NO: 8 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 35, 43, 51, 54, 75, 85, 107, 127, 129, 143, 184, 194, 223, 227, 236, 251, 258 and so on in the nucleotide sequence of SEQ ID NO: 8 can be recited.

To be more specific, when the useful protein gene is a Thrombomodulin gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a Thrombomodulin gene derived from human, and a promoter region located 5′ upstream of the same can be recited, and more concretely, the nucleotide sequence of SEQ ID NO: 9 (corresponding to the nucleotide sequence represented by base No. 1 to 6096 in the nucleotide sequence described in Genbank Accession No. AF495471) can be recited. In the nucleotide sequence of SEQ ID NO: 9, ATG codon encoding methionine at amino terminal of Thrombomodulin protein derived from human is represented in base No. 2590 to 2592, and a nucleotide sequence of the above exon 1 is represented in base No. 2048 to 6096. Cytosine in the nucleotide sequence represented by CpG which is present in the nucleotide sequence of SEQ ID NO: 9, in particular, cytosine in CpG which is present in a region where CpGs are densely present in the nucleotide sequence of SEQ ID NO: 9 exhibits high methylation frequency (namely, a high methylation state (hypermethylation)) in, for example, cancer cells such as gastric cancer cells. More concretely, as cytosine exhibiting high methylation frequency in gastric cancer cells, for example, cytosines represented by base Nos. 1539, 1551, 1571, 1579, 1581, 1585, 1595, 1598, 1601, 1621, 1632, 1638, 1645, 1648, 1665, 1667, 1680, 1698, 1710, 1724, 1726, 1756 and so on in the nucleotide sequence of SEQ ID NO: 9 can be recited.

To be more specific, when the useful protein gene is a p53-responsive gene 2 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a p53-responsive gene 2 gene derived from human, and a promoter region located 5′ upstream of the same can be recited, and more concretely, the nucleotide sequence of SEQ ID NO: 10 (corresponding to a complementary sequence to the nucleotide sequence represented by base No. 113501 to 116000 in the nucleotide sequence described in Genbank Accession No. AC009471) can be recited. In the nucleotide sequence of SEQ ID NO: 10, a nucleotide sequence of exon 1 of a p53-responsive gene 2 gene derived from human is represented in base No. 1558 to 1808. Cytosine in the nucleotide sequence represented by CpG which is present in the nucleotide sequence of SEQ ID NO: 10 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1282, 1284, 1301, 1308, 1315, 1319, 1349, 1351, 1357, 1361, 1365, 1378, 1383 and so on in the nucleotide sequence of SEQ ID NO: 10 can be recited.

To be more specific, when the useful protein gene is a Fibrillin2 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a Fibrillin2 gene derived from human, and a promoter region located 5′ upstream of the same can be recited, and more concretely, the nucleotide sequence of SEQ ID NO: 11 (corresponding to a complementary sequence to the nucleotide sequence represented by base No. 118801 to 121000 in the nucleotide sequence described in Genbank Accession No. AC113387) can be recited. In the nucleotide sequence of SEQ ID NO: 11, a nucleotide sequence of exon 1 of a Fibrillin2 gene derived from human is represented in base No. 1091 to 1345. Cytosine in the nucleotide sequence represented by CpG which is present in the nucleotide sequence of SEQ ID NO: 11 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 679, 687, 690, 699, 746, 773, 777, 783, 795, 799, 812, 823, 830, 834, 843 and so on in the nucleotide sequence of SEQ ID NO: 11 can be recited.

To be more specific, when the useful protein gene is a Neurofilament3 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a Neurofilament3 gene derived from human, and a promoter region located 5′ upstream of the same can be recited, and more concretely, the nucleotide sequence of SEQ ID NO: 12 (corresponding to a complementary sequence to the nucleotide sequence represented by base No. 28001 to 30000 in the nucleotide sequence described in Genbank Accession No. AF106564) can be recited. In the nucleotide sequence of SEQ ID NO: 12, a nucleotide sequence of exon 1 of a Neurofilament3 gene derived from human is represented in base No. 614 to 1694. Cytosine in the nucleotide sequence represented by CpG which is present in the nucleotide sequence of SEQ ID NO: 12 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 428, 432, 443, 451, 471, 475, 482, 491, 499, 503, 506, 514, 519, 532, 541, 544, 546, 563, 566, 572, 580 and so on in the nucleotide sequence of SEQ ID NO: 12 can be recited.

To be more specific, when the useful protein gene is a disintegrin and metalloproteinase domain 23 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a disintegrin and metalloproteinase domain 23 gene derived from human, and a promoter region located 5′ upstream of the same can be recited, and more concretely, the nucleotide sequence of SEQ ID NO: 13 (corresponding to the nucleotide sequence represented by base No. 21001 to 23300 in the nucleotide sequence described in Genbank Accession No. AC009225) can be recited. In the nucleotide sequence of SEQ ID NO: 13, a nucleotide sequence of exon 1 of a disintegrin and metalloproteinase domain 23 gene derived from human is represented in base No. 1194 to 1630. Cytosine in the nucleotide sequence represented by CpG which is present in the nucleotide sequence of SEQ ID NO: 13 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 998, 1003, 1007, 1011, 1016, 1018, 1020, 1026, 1028, 1031, 1035, 1041, 1043, 1045, 1051, 1053, 1056, 1060, 1066, 1068, 1070, 1073, 1093, 1096, 1106, 1112, 1120, 1124, 1126 and so on in the nucleotide sequence of SEQ ID NO: 13 can be recited.

To be more specific, when the useful protein gene is a G protein-coupled receptor 7 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a G protein-coupled receptor 7 gene derived from human, and a promoter region located 5′ upstream of the same can be recited, and more concretely, the nucleotide sequence of SEQ ID NO: 14 (corresponding to a nucleotide sequence represented by base No. 75001 to 78000 in the nucleotide sequence described in Genbank Accession No. AC009800) can be recited. In the nucleotide sequence of SEQ ID NO: 14, a nucleotide sequence of exon 1 of a G protein-coupled receptor 7 gene derived from human is represented in base No. 1666 to 2652. Cytosine in the nucleotide sequence represented by CpG which is present in the nucleotide sequence of SEQ ID NO: 14 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1480, 1482, 1485, 1496, 1513, 1526, 1542, 1560, 1564, 1568, 1570, 1580, 1590, 1603, 1613, 1620 and so on in the nucleotide sequence of SEQ ID NO: 14 can be recited.

To be more specific, when the useful protein gene is a G-protein coupled somatostatin and angiotensin-like peptide receptor gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a G-protein coupled somatostatin and angiotensin-like peptide receptor gene derived from human, and a promoter region located 5′ upstream of the same can be recited, and more concretely, the nucleotide sequence of SEQ ID NO: 15 (corresponding to a complementary sequence to a nucleotide sequence represented by base No. 57001 to 60000 in the nucleotide sequence described in Genbank Accession No. AC008971) can be recited. In the nucleotide sequence of SEQ ID NO: 15, a nucleotide sequence of exon 1 of a G-protein coupled somatostatin and angiotensin-like peptide receptor gene derived from human is represented in base No. 776 to 2632. Cytosine in the nucleotide sequence represented by CpG which is present in the nucleotide sequence of SEQ ID NO: 15 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 470, 472, 490, 497, 504, 506, 509, 514, 522, 540, 543, 552, 566, 582, 597, 610, 612 and so on in the nucleotide sequence of SEQ ID NO: 15 can be recited.

To be more specific, when the useful protein gene is a Solute carrier family 6 neurotransmitter transporter noradrenalin member 2 gene, as a nucleotide sequence that contains at least one nucleotide sequence represented by CpG present in a nucleotide sequence of its promoter region, untranslated region or translated region (coding region), a nucleotide sequence of a genomic DNA containing exon 1 of a Solute carrier family 6 neurotransmitter transporter noradrenalin member 2 gene derived from human, and a promoter region located 5′ upstream of the same can be recited, and more concretely, the nucleotide sequence of SEQ ID NO: 16 (corresponding to a complementary sequence to a nucleotide sequence represented by base No. 78801 to 81000 in the nucleotide sequence described in Genbank Accession No. AC026802) can be recited. In the nucleotide sequence of SEQ ID NO: 16, a nucleotide sequence of exon 1 of a Solute carrier family 6 neurotransmitter transporter noradrenalin member 2 gene derived from human is represented in base No. 1479 to 1804. Cytosine in the nucleotide sequence represented by CpG which is present in the nucleotide sequence of SEQ ID NO: 16 exhibits high methylation frequency (namely, a high methylation state (hypermethylation)) in, for example, cancer cells such as pancreas cancer cells. More concretely, as cytosine exhibiting high methylation frequency in pancreas cancer cells, for example, cytosines represented by base Nos. 1002, 1010, 1019, 1021, 1051, 1056, 1061, 1063, 1080, 1099, 111 0, 1139, 1141, 1164, 1169, 1184 and so on in the nucleotide sequence of SEQ ID NO: 16 can be recited.

In the present invention, the “amount of the amplified DNA (amplifying methylated DNA in the objective DNA region to a detectable level)” means an amount itself after amplification of methylated DNA in the objective region possessed by a genomic DNA contained in the biological specimen, namely, the amount determined in Third step of the present measuring method. For example, when the biological specimen is 1 mL of serum, it means the amount of DNA amplified based on the methylated DNA contained in 1 mL of serum.

The term “methylation rate” in the present invention (particularly, the present methylation rate measuring method) means the numerical value obtained by dividing the amount after amplification of methylated DNA by a total of the amount of methylated DNA after amplification in the objective DNA region possessed by a genomic DNA contained in the biological specimen and the amount of unmethylated DNA after amplification.

The “methylation-sensitive restriction enzyme” in the present invention, for example, a restriction enzyme or the like that does not digest a recognition sequence containing methylated cytosine, but digests only a recognition sequence containing unmethylated cytosine. In other words, in the case of DNA wherein cytosine contained in a recognition sequence inherently recognizable by the methylation sensitive restriction enzyme is methylated, the DNA will not be cleaved even when the methylation sensitive restriction enzyme is caused to act on the DNA. On the other hand, in the case of DNA wherein cytosine contained in a recognition sequence inherently recognizable by the methylation sensitive restriction enzyme is not methylated, the DNA will be cleaved when the methylation sensitive restriction enzyme is caused to act on the DNA. Concrete examples of such methylation sensitive restriction enzymes include HpaII, BstUI, NarI, SacII, and HhaI. The aforementioned methylation sensitive restriction enzyme will not cleave double-stranded DNA containing a CpG pair in a hemimethyl state (namely, double-stranded DNA wherein cytosine in one strand is methylated and cytosine in the other strand is not methylated in the above CpG pair) and this is already revealed by Gruenbaum et al. (Nucleic Acid Research, 9, 2509-2515). Some methylation sensitive restriction enzymes digest single-stranded DNA. Such a restriction enzyme does not digest a recognition sequence containing methylated cytosine in the single-stranded DNA, and is able to digest only a recognition sequence containing non-methylated cytosine. As a methylation sensitive restriction enzyme that digests single-stranded DNA, for example, HhaI and the like can be recited.

The term “masking oligonucleotide” used herein means oligonucleotide having a nucleotide sequence complementary to the nucleotide sequence of the recognition site of the methylation sensitive restriction enzyme, and is oligonucleotide that forms double strand by complementary base-pairing at least one site (even every site is possible) of several recognition sites of the methylation sensitive restriction enzyme contained in the objective DNA region in the single-stranded DNA (that is, the site is made into double-stranded state), thereby enabling the methylation sensitive restriction enzyme that uses only double-stranded DNA as a substrate to digest the site, and improving digestion efficiency at the site for the methylation sensitive restriction enzyme capable of digesting single-stranded DNA (methylation sensitive restriction enzyme capable of digesting single-stranded DNA also digests double-stranded DNA, and digestion efficiency thereof is higher with respect to double-stranded DNA than with respect to single-stranded DNA), and means oligonucleotide not inhibiting formation of double strand between single-stranded DNA containing the objective DNA region and single-stranded immobilized oligonucleotide. Further, the masking oligonucleotide should be oligonucleotide that is unavailable in a reaction for extending an extension primer by using a later-described reverse primer (plus strand) as the extension primer and the masking oligonucleotide (minus strand) as a template. As a nucleotide length, 8 to 200 bases long is preferred.

The masking oligonucleotide to be mixed with the single-stranded DNA containing the objective DNA region (plus strand) contained in a DNA sample derived from genomic DNA may be one kind or plural kinds. When plural kinds are used, many of recognition sites of the methylation sensitive restriction enzyme in the single-stranded DNA containing the objective DNA region become double-strand state, and “DNA remaining undigested” as will be described later by the methylation sensitive restriction enzyme can be minimized. For example, it is particularly useful to use the masking oligonucleotide designed in accordance with a site intended not to be digested when it is methylated and intended to be digested when it is not methylated among several recognition sequences of the methylation sensitive restriction enzyme contained in the objective DNA region (for example, the site that is methylated at 100% in a diseased patient specimen, but is not methylated at 100% in a healthy specimen).

The “single-stranded DNA containing one or more unmethylated CpGs in the recognition site of the methylation sensitive restriction enzyme protected by the masking oligonucleotide” used herein means single-stranded DNA in which cytosine in one or more CpGs present in the recognition site of the restriction enzyme is non-methylated cytosine.

The “extensionally-formed single-stranded DNA not containing unmethylated CpG in the recognition site of the methylation sensitive restriction enzyme protected by the masking oligonucleotide” used herein means single-stranded DNA in which cytosine in every CpG present in the recognition site of the restriction enzyme in single-stranded DNA is methylated.

In First (B) step of combined step (i) and First step of combined step (ii) in the present measuring method, from the DNA sample derived from genomic DNA contained in a biological specimen, the single-stranded DNA (plus strand) containing the objective DNA region (in which recognition site of methylation sensitive restriction enzyme is protected by a masking oligonucleotide) and the single-stranded immobilized oligonucleotide having a nucleotide sequence complementary to a part (provided that, not containing the objective DNA region) of the 3′-end of the single-stranded DNA are base-paired to achieve selection.

In First (B) step of combined step (i) and First step of combined step (ii) in the present measuring method, the “single-stranded immobilized oligonucleotide” is single-stranded immobilized oligonucleotide having a nucleotide sequence complementary to a part (provided that, not containing the objective DNA region) of the 3′-end of the single-stranded DNA (plus strand) containing the objective DNA region (hereinafter, also referred to as present immobilized oligonucleotide).

The present immobilized oligonucleotide is used for selecting the single-stranded DNA (plus strand) containing the objective DNA region from the DNA sample derived from genomic DNA contained in a biological specimen. The present immobilized oligonucleotide is preferably 5 to 50 bases in length.

The 5′-end side of the present immobilized oligonucleotide can be immobilized to a carrier, while the 3′-end thereof should be in a free state for allowing one extension reaction proceeding from the 5′-end to the 3′-end in Second pre step and Step A2 as will be described later. Here, by the expression “the one that can be immobilized to a carrier”, it suffices that the present immobilized oligonucleotide is immobilized to a carrier in selecting the single-stranded DNA (plus strand) containing the objective DNA region, and (1) the one immobilized by binding between the present immobilized oligonucleotide and a carrier at the stage prior to base-pairing between the single-stranded DNA (plus strand) and the present immobilized oligonucleotide, and (2) the one immobilized by binding between the present immobilized oligonucleotide and a carrier at the stage before base-pairing between the single-stranded DNA (plus strand) and the present immobilized oligonucleotide can be recited.

For obtaining such a structure, the 5′-end of the oligonucleotide having the nucleotide sequence complementary to a part (provided that not containing the objective DNA region) of the 3′-end of the single-stranded DNA (plus strand) containing the objective DNA region (hereinafter, also referred to “present oligonucleotide”) may be immobilized to a carrier according to a general genetic engineering method or commercially available kit, apparatus and the like (binding to solid phase). A concrete exemplary method involves biotinylating the 5′-end of the present oligonucleotide, and immobilizing the obtained biotinylated oligonucleotide to a support coated with streptavidin (for example, a PCR tube coated with streptavidin, magnetic beads coated with streptavidin and so on).

Also, such a method can be recited that after covalently binding a molecule having an active functional group such as an amino group, an aldehyde group or a thiol group to 5′-end side of the present oligonucleotide, the product is covalently bound to a support made of glass, silica or heat-resistant plastic having a surface activated with a silane coupling agent or the like, via a spacer, a cross linker or the like such as five serially-connected triglycerides. Also, a method of chemically synthesizing from the 5′-end side of the present oligonucleotide directly on a support made of glass or silicon is recited.

In First (A) step of combined step (i) and Second (A) step of combined step (ii) in the present measuring method, single-stranded DNA (plus strand) containing the objective DNA region is mixed with masking oligonucleotide having a nucleotide sequence complementary to the nucleotide sequence of the recognition site of the methylation sensitive restriction enzyme.

By mixing the masking oligonucleotide, as previously described, it is possible to protect at least one site (even every site is possible) of several recognition sites of the methylation sensitive restriction enzyme contained in the objective DNA region in the single-stranded DNA (that is, the site is made into double-stranded state), thereby enabling the methylation sensitive restriction enzyme that uses only double-stranded DNA as a substrate to digest the site, and improving digestion efficiency at the site by the methylation sensitive restriction enzyme capable of digesting single-stranded DNA (methylation sensitive restriction enzyme capable of digesting single-stranded DNA also digests double-stranded DNA, and digestion efficiency thereof is higher with respect to double-stranded DNA than with respect to single-stranded DNA). In other words, prior to a digestion treatment by the methylation sensitive restriction enzyme in Second step of combined step (i) and Second (B) step of combined step (ii) in the present measuring method, the masking oligonucleotide is base-paired with the recognition site of the methylation sensitive restriction enzyme contained in the objective DNA region of the single-stranded DNA, to enable the methylation sensitive restriction enzyme that uses double-stranded DNA as a substrate to digest the recognition sequence of the methylation sensitive restriction enzyme having unmethylated CpG. The masking oligonucleotide may be mixed before or after separating genomic DNA into single strand, and the masking oligonucleotide may be mixed in First (A) step of combined step (i), and further, the masking oligonucleotide may be mixed in Second (A) step of combined step (ii). In brief, it is base-paired with a nucleotide sequence that can be recognized by the methylation sensitive restriction enzyme to form a double-stranded state before treatment with the methylation sensitive restriction enzyme.

In Second step of combined step (i) and Second (B) step of combined step (ii) in the present measuring method, the single-stranded DNA selected in First (B) step of combined step (i) or First step of combined step (ii) is digested with one or more kinds of methylation sensitive restriction enzyme, and then a generated free digest (single-stranded DNA containing one or more unmethylated CpGs in the recognition site of the methylation sensitive restriction enzyme protected by the masking oligonucleotide) is removed.

As a method of examining whether or not digestion by the methylation sensitive restriction enzyme occurs, concretely, for example, a method of conducting PCR using a pair of primers capable of amplifying DNA containing cytosine which is a target of analysis in a recognition sequence while using the DNA as a template, and examining whether or not the DNA is amplified (amplified product) can be recited. When the cytosine which is a target of analysis is methylated, an amplified product is obtained. On the other hand, when the cytosine which is a target of analysis is not methylated, an amplified product is not obtained. In this manner, by comparing the amounts of amplified DNA, it is possible to measure the methylated rate of the cytosine which is a target of analysis.

By the way, in the single-stranded DNA selected in First (B) step of combined step (i) or First step of combined step (ii), since masking oligonucleotide is added, the part base-paired with single-stranded immobilized oligonucleotide and the objective DNA region with which the masking oligonucleotide is base-paired are in a double-stranded state. While the present immobilized oligonucleotide as a minus strand fails to be base-paired with the objective DNA region, the recognition site of the methylation sensitive restriction enzyme is base-paired with the masking oligonucleotide to be in a double-stranded state. Cytosine contained in the masking oligonucleotide as a minus strand is in a non-methylated state, and whether or not the single-strand DNA is in an unmethylated state is determined depending on whether cytosine contained in the single-stranded DNA of genomic DNA contained in a biological specimen is methylated or non-methylated. In other words, when genomic DNA contained in a biological specimen is methylated, the double-stranded DNA part with which the masking oligonucleotide is base-paired is in a hemimethyl state (the state that is not an unmethylated state, minus strand: a non-methylated state, plus strand: a methylated state), and when genomic DNA contained in a biological specimen is not methylated, the double-stranded DNA part with which the masking oligonucleotide is base-paired is in an unmethylated state (minus strand: a non-methylated state, plus strand: a non-methylated state). Therefore, by utilizing a characteristic that the aforementioned methylation sensitive restriction enzyme does not cleave double-stranded DNA in a hemimethyl state, it is possible to distinguish whether cytosine in one or more CpG pairs present in the recognition site of the methylation sensitive restriction enzyme in genomic DNA in a biological specimen is methylated or not. That is, by conducting a digestion treatment with the methylation sensitive restriction enzyme, if cytosine in one or more CpG pairs present in the double-stranded DNA part with which the masking oligonucleotide is base-paired in genomic DNA contained in a biological specimen is not methylated, the double-stranded DNA part with which the masking oligonucleotide is base-paired is in an unmethylated state, and cleaved by the methylation sensitive restriction enzyme. If cytosine in every CpG pair present in the double-stranded DNA part with which the masking oligonucleotide is base-paired in genomic DNA contained in a biological specimen is methylated, the double-stranded DNA part with which the masking oligonucleotide is base-paired is in a hemimethyl state, and will not be cleaved by the methylation sensitive restriction enzyme.

Therefore, as a result of PCR using a pair of primers capable of amplifying the objective DNA region after conducting a digestion treatment following base-pairing between the nucleotide sequence of the recognition site of the methylation sensitive restriction enzyme contained in the objective DNA region and the masking oligonucleotide, an amplified product will not be obtained if cytosine in one or more CpG pairs present in the double-stranded DNA part with which the masking oligonucleotide is base-paired in genomic DNA contained in a biological specimen is not methylated, whereas an amplified product will be obtained if cytosine in every CpG pair present in the double-stranded DNA part with which the masking oligonucleotide is base-paired in genomic DNA contained in a biological specimen is methylated.

To be more specific, for example, by using HpaII or HhaI as a methylation sensitive restriction enzyme, and using masking oligonucleotide, it is possible to distinguish whether or not the single-stranded DNA is methylated. That is, if cytosine of CpG contained in the recognition site of HpaII or HhaI in the single-stranded DNA in which the single-stranded immobilized oligonucleotide is base-paired obtained in the above operation is methylated, HpaII or HhaI is not able to digest the DNA. On the other hand, if it is not methylated, HpaII or HhaI is able to digest the DNA. Therefore, when a PCR reaction is conducted using a pair of primers capable of amplifying the objective DNA region after conducting the above reaction, an amplified product will not be obtained if DNA in the objective DNA region possessed by genomic DNA contained in a biological specimen is not methylated, whereas an amplified product will be obtained if the DNA is methylated.

The process up to Second step of combined step (i) of the present measuring method is concretely executed, for example, in the following manner in a case where the present immobilized oligonucleotide is biotinylated oligonucleotide.

(a) First, a DNA sample derived from genomic DNA contained in a biological specimen is mixed with an annealing buffer and masking oligonucleotide having a nucleotide sequence complementary to the nucleotide sequence of a recognition site of a methylation sensitive restriction enzyme.

(b) Then, the obtained mixture is heated at 95° C. for several minutes for making double-stranded DNA containing an objective DNA region present in the DNA sample derived from genomic DNA contained in a biological specimen into single-stranded DNA. Thereafter, in order to form single-stranded DNA in which the recognition site of the methylation sensitive restriction enzyme is protected containing the objective DNA region (for forming a partly double strand with masking oligonucleotide), for example, the mixture is rapidly cooled to the temperature lower than Tm value of the masking oligonucleotide by about 0 to 20° C., and kept at that temperature for several minutes.

(c) To the sample obtained in the above is added with biotinylated oligonucleotide (presently in a free state because it is immobilized by binding between the present immobilized oligonucleotide and a carrier after base-paring between the single-stranded DNA (plus strand) and the present immobilized oligonucleotide) to obtain a mixture.

(d) Then, the obtained mixture is heated at 95° C. for several minutes. Then, the mixture is rapidly cooled to the temperature lower than Tm value of the masking oligonucleotide by about 0 to 20° C., and kept at that temperature for several minutes, for example, for allowing base-pairing between the single-stranded DNA (plus strand) containing the objective DNA region and the biotinylated oligonucleotide.

(e) By adding the mixture obtained in the above (d) to a support coated with streptavidin, and keeping it at 37° C. for several minutes, the biotinylated oligonucleotide is immobilized to the support coated with streptavidin.

While base-pairing between the single-stranded DNA containing the objective DNA region (plus strand) and the biotinylated oligonucleotide is executed prior to immobilization of the biotinylated oligonucleotide and the support coated with streptavidin in the above (a) to (e), as described above, the order may be reverse.

Also, the operation of (c) or later may be executed without conducting (b) after execution of the above (a), or the operation of (e) may be executed without conducting (d) after execution of the operation up to the above (c).

(f) After immobilizing the biotinylated oligonucleotide to the support coated with streptavidin, removal and washing of the remaining solution (DNA purification) are conducted.

More concretely, for example, when a PCR tube coated with streptavidin is used, after removing the solution by pipetting or decantation first, a wash buffer of a volume approximately equal to the volume of the biological specimen is added, and thereafter, the wash buffer may be removed by pipetting or decantation. When magnetic beads coated with streptavidin are used, after immobilizing the beads with a magnet, the solution is removed by pipetting or decantation first, and a wash buffer of a volume approximately equal to the volume of the biological specimen is added, and thereafter, the wash buffer may be removed by pipetting or decantation.

Then, by executing these operations several times, the remaining solution is removed and washed (DNA purification).

These operations are important for removing unimmobilized DNA, or DNA floating in the solution digested with the restriction enzyme as will be described later, from the reaction solution. If these operations are inadequate, the DNA floating in the reaction solution will be a template and an unexpected amplification product will be obtained by an amplification reaction. In order to avoid non-specific binding between the support and DNA in the biological specimen, the above operations may be executed while a large amount of DNA having a nucleotide sequence which is completely different from that of the objective region (for example, rat DNA and so on, in the case of a human biological specimen) is added to the biological specimen.

(g) The sample obtained in the above (f) is added with one or more kinds of methylation sensitive restriction enzyme, and incubated at 37° C. for 1 hour to overnight to achieve a digestion treatment. Concretely, for example, the operation may be conducted in the following manner. The sample obtained in the above (e) is added with 3 μL of an optimum buffer (330 mM Tris-Acetate pH 7.9, 660 mM KOAc, 100 mM MgOAc2, 5 mM Dithiothreitol), each 1.5 μL of methylation sensitive restriction enzyme HpaII, HhaI (10 U/μL) or the like, and an appropriate amount of BSA or the like as necessary, and then the resultant mixture is added with sterilized ultrapure water to make the liquid volume 30 μL, and incubated at 37° C. for 1 hour to overnight. As a result, when the recognition site of the methylation sensitive restriction enzyme (site) protected by masking oligonucleotide contained in the objective DNA region is not methylated, the site will be digested.

(h) After a digestion treatment with one or more kinds of methylation sensitive restriction enzyme in this manner, removal and washing of the generated free digest (single-stranded DNA containing one or more unmethylated CpGs in the recognition site of the methylation sensitive restriction enzyme, the site being protected by the masking oligonucleotide) (DNA purification) are conducted. To be more specific, for example, when a PCR tube coated with streptavidin is used, after removing the solution by pipetting or decantation at first, a washing buffer of an amount approximately equal to the volume of the biological specimen is added, and thereafter, the washing buffer may be removed by pipetting or decantation. When magnetic beads coated with streptavidin are used, after immobilizing the beads by magnet, the solution is removed by pipetting or decantation at first, and a washing buffer of an amount approximately equal to the volume of the biological specimen is added, and thereafter, the washing buffer may be removed by pipetting or decantation. Then, by executing these operations several times, removal and washing of the digest (single-stranded DNA containing one or more unmethylated CpGs in recognition site of the restriction enzyme) (DNA purification) are executed.

The process up to Second step of combined step (ii) of the present measuring method is concretely executed, for example, in the following manner in a case where the present immobilized oligonucleotide is biotinylated oligonucleotide.

(a) First, a DNA sample derived from genomic DNA contained in a biological specimen is added with an annealing buffer and biotinylated oligonucleotide (presently in a free state because it is immobilized by binding between the present immobilized oligonucleotide and a carrier after base-paring between single-stranded DNA (plus strand) and the present immobilized oligonucleotide) to obtain a mixture.

(b) Next, the obtained mixture is heated at 95° C. for several minutes in order to make double-stranded DNA containing the objective DNA region present in a DNA sample derived from genomic DNA contained in a biological specimen into a single strand. Thereafter, for allowing base-pairing between the single-stranded DNA (plus strand) containing the objective DNA region and biotinylated oligonucleotide, for example, the mixture is rapidly cooled to the temperature lower than Tm value of masking oligonucleotide by about 0 to 20° C., and kept at that temperature for several minutes.

(c) The biotinylated oligonucleotide is immobilized to a support coated with streptavidin by adding the mixture obtained in the above (d) to the support coated with streptavidin, and keeping it at 37° C. for several minutes.

By the way, as described above, in the above (a) to (c), base-pairing between the single-stranded DNA (plus strand) containing an objective DNA region, and the biotinated oligonucleotide is executed in an earlier stage than immobilization of the biotinated oligonucleotide on the support coated with streptavidin, however, the order may be inverted.

(d) After immobilizing the biotinylated oligonucleotide to the support coated with streptavidin, removal and washing of the remaining solution (DNA purification) are conducted.

More concretely, for example, when a PCR tube coated with streptavidin is used, after removing the solution by pipetting or decantation first, a wash buffer of a volume approximately equal to the volume of the biological specimen is added, and thereafter, the wash buffer may be removed by pipetting or decantation. When magnetic beads coated with streptavidin are used, after immobilizing the beads with a magnet, the solution is removed by pipetting or decantation first, and a wash buffer of a volume approximately equal to the volume of the biological specimen is added, and thereafter, the wash buffer may be removed by pipetting or decantation.

Then, by executing these operations several times, the remaining solution is removed and washed (DNA purification).

These operations are important for removing unimmobilized DNA, or DNA floating in the solution digested with the restriction enzyme as will be described later, from the reaction solution. If these operations are inadequate, the DNA floating in the reaction solution will be a template and an unexpected amplification product will be obtained by an amplification reaction. In order to avoid non-specific binding between the support and DNA in the biological specimen, the above operations may be executed while a large amount of DNA having a nucleotide sequence which is completely different from that of the objective region (for example, rat DNA and so on, in the case of a human biological specimen) is added to the biological specimen.

(e) The sample obtained in the above (d) is added with masking oligonucleotide and one or more kinds of methylation sensitive restriction enzyme, and incubated at 37° C. for 1 hour to overnight to achieve a digestion treatment. In the present operation, Second (A) step and Second (B) step can be executed simultaneously. Concretely, for example, the operation may be executed in the following manner. The sample obtained in the above (d) is added with 3 μL of an optimum buffer (330 mM Tris-Acetate pH 7.9, 660 mM KOAc, 100 mM MgOAc2, 5 mM Dithiothreitol), each 1.5 μL of methylation sensitive restriction enzyme HpaII, HhaI (10 U/μL) or the like, each about 10 pmol of the masking oligonucleotide for the recognition sequence of the methylation sensitive restriction enzyme, and an appropriate amount of BSA or the like as necessary, and then the resultant mixture is added with sterilized ultrapure water to make the liquid volume 30 μL, and incubated at 37° C. for 1 hour to overnight. As a result, when the recognition site of the methylation sensitive restriction enzyme (site) protected by masking oligonucleotide contained in the objective DNA region is not methylated, the site will be digested.

(f) After a digestion treatment with one or more kinds of methylation sensitive restriction enzyme in this manner, removal and washing of the generated free digest (single-stranded DNA containing one or more unmethylated CpGs in the recognition site of the methylation sensitive restriction enzyme the site being protected by the masking oligonucleotide) (DNA purification) are conducted. More concretely, for example, when a PCR tube coated with streptavidin is used, after removing the solution by pipetting or decantation first, a wash buffer of a volume approximately equal to the volume of the biological specimen is added, and thereafter, the wash buffer may be removed by pipetting or decantation. When magnetic beads coated with streptavidin are used, after immobilizing the beads with a magnet, the solution is removed by pipetting or decantation first, and a wash buffer of a volume approximately equal to the volume of the biological specimen is added, and thereafter, the wash buffer may be removed by pipetting or decantation.

Then, by executing these operations several times, the digest (single-stranded DNA containing one or more unmethylated CpG pair in the recognition site of the restriction enzyme) is removed and washed (DNA purification).

As a preferred aspect in base-pairing between single-stranded DNA (plus strand) containing the objective DNA region, and single-stranded immobilized oligonucleotide having a nucleotide sequence complementary to a part (provided that, not containing the objective DNA region) of the 3′-end of the single-stranded DNA, and in base pairing between single-stranded DNA (plus strand) containing the objective DNA region and masking oligonucleotide up to Second step of combined steps (i) and (ii) in the present measuring method, base-pairing in a reaction system containing a divalent cation can be recited. More preferably, the divalent cation is a magnesium ion. The “reaction system containing a divalent cation” used herein means a reaction system that contains a divalent cation in an annealing buffer used for base-pairing between the single-stranded DNA (plus strand) and the single-stranded immobilized oligonucleotide, and concretely and preferably contains a salt containing magnesium ions (for example, MgOAc₂, MgCl₂ and so on) in a concentration of 1 mM to 600 mM.

As a concern in a digestion treatment by the methylation sensitive restriction enzyme in Second step of combined step (i) and Second (B) step of combined step (ii) in the present measuring method, a fear that a recognition sequence containing non-methylated cytosine cannot be completely digested (so called “DNA remaining undigested”) can be recited. When such a fear is problematic, since the “DNA remaining undigested” can be minimized if recognition sites of the methylation sensitive restriction enzyme abundantly exist, it is considered that as the objective DNA region, the one having one or more recognition sites of the methylation sensitive restriction enzyme is preferred.

Therefore, when a treatment with a plurality of methylation sensitive restriction enzymes is executed in Second step of combined step (i) and Second (B) step of combined step (ii) in the present measuring method, concretely the operation may be conducted in the following manner. The single-stranded DNA selected in First (B) step of combined step (i) or Second (A) step of combined step (ii) is added with 3 μL of an optimum buffer (330 mM Tris-Acetate pH 7.9, 660 mM KOAc, 100 mM MgOAc2, 5 mM Dithiothreitol), each 1.5 μL of one or more kinds of methylation sensitive restriction enzyme HpaII and/or HhaI (10 U/μL), and an appropriate amount of BSA or the like as necessary, and then the resultant mixture is added with sterilized ultrapure water to make the liquid volume 30 μL, and incubated at 37° C. for 1 hour to overnight. As a result, when the recognition site of the methylation sensitive restriction enzyme (site) protected by masking oligonucleotide contained in the objective DNA region is not methylated, the site will be digested. Then, according to a similar operation as described above, removal and washing of the remaining solution (DNA purification) are conducted by pipetting or decantation. More concretely, for example, when a PCR tube coated with streptavidin is used, after removing the solution by pipetting or decantation first, a wash buffer of a volume approximately equal to the volume of the biological specimen is added, and thereafter, the wash buffer may be removed by pipetting or decantation. When magnetic beads coated with streptavidin are used, after immobilizing the beads with a magnet, the solution is removed by pipetting or decantation first, and a wash buffer of a volume approximately equal to the volume of the biological specimen is added, and thereafter, the wash buffer may be removed by pipetting or decantation.

In the present measuring method or a methylation rate measuring method as will be described later, one preferable embodiment is that “a DNA sample derived from a genomic DNA contained in a biological specimen” is a DNA sample digested in advance with a restriction enzyme whose recognition cleavage site excludes the objective DNA region possessed by the genomic DNA. Here, when a genomic DNA contained in a biological specimen (template DNA) is selected with the use of present immobilized oligonucleotide, shorter template DNA is more likely to be selected, and when the objective region is amplified by PCR, shorter template DNA is more preferred. Therefore, a digestion treatment may be executed while using a restriction enzyme whose recognition cleavage site excludes the objective DNA region directly on the DNA sample derived from a genomic DNA contained in a biological specimen. As a method of digesting with a restriction enzyme whose recognition cleavage site excludes the objective DNA region, a commonly used restriction enzyme treatment method may be used.

One exemplary preferable embodiment is that “a DNA sample derived from a genomic DNA contained in a biological specimen” is a DNA sample digested with one or more kinds of methylation sensitive restriction enzyme.

These embodiments are preferred because the methylation amount can be determined accurately by digesting the biological specimen itself in advance with a restriction enzyme as described above. Such a method is useful for avoiding the “DNA remaining undigested” as described above.

As a method of digesting a sample derived from a genomic DNA contained in a biological specimen with the methylation sensitive restriction enzyme, when the biological specimen is a genomic DNA itself, the method similar to that described above is preferred, and when the biological specimen is a tissue lysate, a cell lysate or the like, a digestion treatment may be executed using a large excess of methylation sensitive restriction enzyme, for example, a methylation sensitive restriction enzyme in an amount of 500 times (10 U) or more with respect to 25 ng of the DNA amount, according to a similar method as described above.

Genomic DNA exists as double-stranded DNA. Therefore, in the present operation, not only a methylation sensitive restriction enzyme (for example, HhaI) capable of digesting single-stranded DNA, but also a methylation sensitive restriction enzyme capable of digesting double-stranded DNA (for example, HpaII, BstUI, NarI, SacII, HhaI and the like) may be used.

In Third step of the present measuring method, as a pre step of each of the following regular steps, the following steps are comprised:

a step of temporarily separating single-stranded DNA which is an undigested substance obtained in Second step (single-stranded DNA not containing unmethylated CpG in the recognition site of a methylation sensitive restriction enzyme the site being protected by masking oligonucleotide) from both of single-stranded immobilized oligonucleotide and masking oligonucleotide (First pre step), and

a step (Second pre step) having

a step of selecting generated DNA in a single strand state by allowing base-pairing between the generated single-stranded DNA (plus strand) and single-stranded oligonucleotide, thereby forming DNA in which the selected single-stranded DNA and the single-stranded oligonucleotide are base-paired (Second (A) pre step), and

a step of making the DNA formed in the step (Second (A) pre step) into double-stranded DNA in which the selected single-stranded DNA has been extended by allowing one extension of a primer by using the selected single-stranded DNA as a template and the single-stranded oligonucleotide as a primer (Second (B) pre step), and

a step of temporarily separating double-stranded DNA extensionally-formed in Second pre step (extensionally-formed double-stranded DNA not containing an unmethylated CpG pair in the recognition site of the methylation sensitive restriction enzyme, the site being protected by the masking oligonucleotide) into a single-stranded DNA (plus strand) and a single-stranded DNA (minus strand) (Third pre step), and as regular steps:

(a) Regular step A having Step A1 of selecting the DNA in a single strand state by allowing base-pairing between the generated single-stranded DNA (plus strand) and the single-stranded immobilized oligonucleotide (minus strand), and Step A2 of extensionally-forming double-stranded DNA from the single-stranded DNA by allowing one extension of a primer by using the single-stranded DNA selected in Step A1 as a template and the single-stranded immobilized oligonucleotide as a primer, and

(b) Regular step B of extensionally-forming double-stranded DNA from the single-stranded DNA by allowing one extension of an extension primer by using the generated single-stranded DNA (minus strand) as a template, and the extension primer (reverse primer) having a nucleotide sequence (plus strand) complementary to a partial nucleotide sequence (minus strand) of a nucleotide sequence possessed by the single-stranded DNA (minus strand) and a partial nucleotide sequence (minus strand) positioned on the 3′-end side than the 3′-end of the nucleotide sequence (minus strand) complementary to the nucleotide sequence (plus strand) of the objective DNA region as an extension primer, and the methylated DNA in the objective DNA region is amplified to a detectable level by repeating each regular step after temporarily separating the extensionally-formed double-stranded DNA obtained in each regular step into a single strand state, and the amplified DNA is quantified.

In Third step of the present measuring method, first, as First pre step among pre steps of respective regular steps, the single-stranded DNA which is an undigested substance obtained in Second step (single-stranded DNA not containing an unmethylated CpG pair in the recognition site of the methylation sensitive restriction enzyme, the site being protected by the masking oligonucleotide) is temporarily separated from the single-stranded immobilized oligonucleotide and the masking oligonucleotide, and is made into a single strand state. Concretely, for example, the single-stranded DNA which is an undigested substance obtained in Second step (single-stranded DNA not containing an unmethylated CpG pair in the recognition site of the methylation sensitive restriction enzyme, the site being protected by the masking oligonucleotide) is added with an annealing buffer to obtain a mixture. Then, the obtained mixture is heated at 95° C. for several minutes. Thereafter, Second (A) pre step may be executed, for example, in accordance with First step of combined step (ii), and DNA made up of methylated single-stranded DNA and single-stranded oligonucleotide that are base-paired is formed. When the single-stranded oligonucleotide is immobilized oligonucleotide, Second (B) step may be executed concretely in the following manner, for example.

The formed DNA is added with 17.85 μL of sterilized ultrapure water, 3 μL of an optimum buffer (100 mM Tris-HCl pH 8.3, 500 mM KCl, 15 mM MgCl₂), 3 μL of 2 mM dNTP, and 6 μL of 5N betaine, and then the resultant mixture is added with 0.15 μL of AmpliTaq (a kind of DNA polymerase: 5 U/μL) to make the liquid volume 30 μL, followed by incubation at 37° C. for 2 hours. Thereafter, the incubated solution is removed by pipetting or decantation, and a washing buffer of an amount substantially equal to the volume of the biological specimen is added, and then the washing buffer may be removed by pipetting or decantation. Then, by executing such operations several times, removal and washing of the remaining solution (DNA purification) are achieved.

The extensionally-formed double-stranded DNA obtained in Second pre step (extensionally-formed double-stranded DNA not containing an unmethylated CpG pair in the recognition site of the methylation sensitive restriction enzyme, the site being protected by the masking oligonucleotide) is temporarily separated into a single-stranded DNA (plus strand) and a single-stranded DNA (minus strand). Concretely, for example, the extensionally-formed double-stranded DNA obtained in Second pre step (extensionally-formed double-stranded DNA not containing an unmethylated CpG pair in the recognition site of the methylation sensitive restriction enzyme, the site being protected by the masking oligonucleotide) is added with an annealing buffer to obtain a mixture.

Then, the obtained mixture is heated at 95° C. for several minutes. Then, the following regular steps are conducted.

(a) The generated single-stranded DNA (plus strand) is rapidly cooled to the temperature lower by about 10 to 20° C. than Tm of single-stranded immobilized oligonucleotide (minus strand), and kept at this temperature for several minutes for allowing annealing with the single-stranded immobilized oligonucleotide (minus strand).

(b) Thereafter, the temperature is restored to room temperature (Step A1 in Regular step A).

(c) Double-stranded DNA is extensionally formed from the single-stranded DNA by one extension of a primer by using the single-stranded DNA selected in the above (a) as a template and the single-stranded immobilized oligonucleotide as the primer (that is, Step A2 in Regular step A). Concretely, for example, it may be practiced in accordance with the operation method or the like in the extension reaction as will be described later or in Second (B) pre step of the present measuring method as described above.

(d) Double-stranded DNA is extensionally formed from the single-stranded DNA by allowing one extension of an extension primer by using the generated single-stranded DNA (minus strand) as a template, and the extension primer (reverse primer) having a nucleotide sequence (plus strand) complementary to the partial nucleotide sequence (minus strand) of a nucleotide sequence possessed by the single-stranded DNA (minus strand) and the partial nucleotide sequence (minus strand) positioned on the 3′-end side than the 3′-end of the nucleotide sequence (minus strand) complementary to the nucleotide sequence (plus strand) of the objective DNA region as an extension primer (that is, Regular step B). Concretely, for example, similarly to the above (c), it may be practiced in accordance with the operation method or the like in the extension reaction as will be described later or in Second (B) pre step of the present measuring method as described above.

(e) Methylated DNA in the objective DNA region is amplified to a detectable level by repeating each regular step (for example Step A and Step B) after temporarily separating the extensionally-formed double-stranded DNA obtained in each regular step into a single strand state, and the amplified DNA is quantified. Concretely, for example, similarly to the above description, it may be practiced in accordance with the operation method or the like as will be described later or in Second (B) pre step, Regular step A and Regular step B in Third step of the present measuring method as described above.

In Third step, concretely the reactions starting from First pre step and up to the regular step may be executed as a single PCR reaction. Also, each reaction from First pre step to Third pre step may be independently executed, and only a regular step may be executed as a PCR reaction.

As a method of amplifying the objective DNA region (that is, objective region) after a digestion treatment with a methylation sensitive restriction enzyme (after completion of Second step), for example, PCR may be used. Since the present immobilized oligonucleotide can be used as one of the primers in amplifying the objective region, an amplification product can be obtained by conducting PCR while adding only the other of the primers, and its amplification product is also immobilized. At this time, by using a primer labeled in advance with fluorescence or the like and utilizing the label as an index, it is possible to evaluate presence or absence of the amplification product without executing a burdensome operation such as electrophoresis. As a PCR reaction solution, for example, a reaction solution prepared by mixing the DNA obtained in Second step of the present measuring method with 0.15 μL of a 50 μM primer solution, 2.5 μL of 2 mM dNTP, 2.5 μL of a buffer (100 mM Tris-HCl pH 8.3, 500 mM KCl, 20 mM MgCl₂, 0.01% Gelatin), and 0.2 μL of AmpliTaq Gold (one kind of thermostable DNA polymerase: 5 U/μL), and adding sterilized ultrapure water to make the liquid volume 25 μL can be recited.

Since an objective DNA region (namely, an objective region) often has a GC rich nucleotide sequence, the reaction may sometimes be executed while adding an appropriate amount of betaine, DMSO or the like. In one exemplary reaction conditions, the reaction solution as described above is retained at 95° C. for 10 minutes, and then a cycle made up of 30 seconds at 95° C., 30 seconds at 55 to 65° C., and 30 seconds at 72° C. is repeated 30 to 40 cycles. After conducting such PCR, the obtained amplification product is detected. For example, when a primer labeled in advance is used, after executing washing and purification operations similar to those as described above, an amount of an immobilized fluorescent label may be measured. When PCR is conducted using a normal primer that is not labeled, a probe or the like that is labeled with gold colloid particles, fluorescence or the like is caused to anneal, and detection may be achieved by measuring an amount of the probe bound to the objective region. Also, in order to determine an amount of the amplification product more accurately, for example, a real-time PCR method may be used. The real-time PCR is a method in which PCR is monitored in real time, and the obtained monitor result is analyzed kinetically, and is known as a high-accuracy quantitative PCR method capable of detecting a very small difference as small as twice the gene amount. As such a real-time PCR method, for example, a method using a probe such as a template-dependent nucleic acid polymerase probe, a method of using an intercalator such as SYBR-Green and the like can be recited. As an apparatus and a kit for the real-time PCR method, those commercially available may be used. As described above, detection may be executed by any method well-known heretofore without any particular limitation. Such methods make it possible to conduct the operations up to detection without changing the reaction container.

Further, the objective region can be amplified by using a biotinylated oligonucleotide having the same nucleotide sequence as single-stranded immobilized oligonucleotide as one of the primers, or biotinylated oligonucleotide newly designed on the 3′-end side from the single-stranded immobilized oligonucleotide as one of the primers, and a primer of a complementary side. In this case, since the obtained amplification product is immobilized if there is a support coated with streptavidin, for example, when PCR is executed in a PCR tube coated with streptavidin, use of labeled primer as described above will facilitate the detection of the amplification product because it will be immobilized inside the tube. When the foregoing single-stranded oligonucleotide is immobilized by covalent bonding or the like, the solution containing an amplification product obtained by PCR may be transferred to a container where a support coated with streptavidin is present, and the amplification product may be immobilized. The detection may be executed in the manner as described above. The complementary side primer for amplifying an objective region should be a primer that is capable of amplifying an objective region having one or more recognition sites of a methylation sensitive restriction enzyme and not containing the recognition site. The reason is as follows. When only the most 3′-end side recognition site of the methylation sensitive restriction enzyme of a DNA chain (newly-generated strand) on the present immobilized oligonucleotide side of the double-stranded DNA obtained by selection and one extension reaction is not methylated, only that part will be digested by the methylation sensitive restriction enzyme. Even if washing operation is conducted as described above after digestion, double-stranded DNA in which a part of the 3′-end in newly-generated strand is lost remains immobilized. When the complementary side primer contains the most 3′-end side recognition site of the methylation sensitive restriction enzyme, several bases on the 3′-end side of the primer anneal with several bases on the 3′-end side of the newly-generated strand, so that the objective region can be amplified by PCR.

The present invention also provides a modified method further comprising a step of adding into a reaction system single-stranded oligonucleotide (minus strand) having a nucleotide sequence complementary to a part (provided that, not containing the objective DNA region) of the 3′-end of single-stranded DNA containing an objective DNA region (plus strand) and is in a free state (Additional pre step), before or after First pre step in Third step of the present measuring method, or before or after Third pre step in Third step.

### That is,

### (Modified Method 1)

A method further comprising a step (Additional pre step) of adding single-stranded oligonucleotide (minus strand) in a free state having a nucleotide sequence complementary to a part (provided that not containing the objective DNA region) of the 3′-end of single-stranded DNA (plus strand) containing the objective DNA region into a reaction system, prior to First pre step in Third step of the present measuring method, and additionally comprising the following one step as Second pre step and a respective regular step in Third step of the present measuring method:

(c) Regular step C having:

(i) Step C1 of selecting single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as the primer.

### (Modified Method 2)

A method further comprising a step (Additional step) of adding single-stranded oligonucleotide (minus strand) in a free state having a nucleotide sequence complementary to a part (provided that not containing the objective DNA region) of the 3′-end of single-stranded DNA (plus strand) containing the objective DNA region into a reaction system, after First pre step in Third step of the present measuring method, and further comprising the following one step as Second pre step and a respective regular step in Third step of the present measuring method (hereinafter, also referred to as the present methylation rate measuring method):

(c) Regular step C having:

(i) Step C1 of selecting the single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as the primer.

### (Modified Method 3)

A method further comprising a step (Additional pre step) of adding into a reaction system single-stranded oligonucleotide (minus strand) in a free state having a nucleotide sequence complementary to a part (provided that not containing the objective DNA region) of the 3′-end of single-stranded DNA (plus strand) containing the objective DNA region, prior to Third pre step in Third of the present measuring method, and further comprising the following one step as Second pre step and a respective regular step in Third step of the present measuring method:

(c) Regular step C having:

(i) Step C1 of selecting single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as the primer.

### (Modified Method 4)

As method further comprising a step (Additional pre step) of adding into a reaction system single-stranded oligonucleotide (minus strand) in a free state having a nucleotide sequence complementary to a part (provided that not containing the objective DNA region) of the 3′-end of single-stranded DNA (plus strand) containing the objective DNA region, after Third pre step in Third step of the present measuring method, and further comprising the following one step as Second pre step and a respective regular step in Third step of the present measuring method:

(c) Regular step C having:

(i) Step C1 of selecting single-stranded DNA by base-paring the generated single-stranded DNA (plus strand) and the single-stranded oligonucleotide (minus strand) added into the reaction system in Additional pre step, and

(ii) Step C2 of making the single-stranded DNA into extensionally-formed double-stranded DNA by one extension of a primer by using the single-stranded DNA selected in Step C1 as a template and the single-stranded oligonucleotide (minus strand) as the primer.

In the modified method, it is possible to readily improve the amplification efficiency of the objective DNA region in Third step by, for example, adding “single-stranded oligonucleotide (minus strand) having a nucleotide sequence complementary to a part (not containing the objective DNA region) of the 3′-end of single-stranded DNA (plus strand) containing an objective DNA region and is in a free state” to a reaction system externally. The single-stranded oligonucleotide (minus strand) added into the reaction system in Additional pre step may have the same nucleotide sequence as that of the single-stranded immobilized oligonucleotide, or may have a shorter nucleotide sequence or a longer nucleotide sequence insofar as it is single-stranded oligonucleotide having a nucleotide sequence complementary to a part (not containing the objective DNA region) of the 3′-end of single-stranded DNA and has the 5′-end which is as same as the single-stranded immobilized oligonucleotide and in a free state. However, in the case of a nucleotide sequence longer than the single-stranded immobilized oligonucleotide, it is important to be single-stranded oligonucleotide in a free state which is unavailable in reaction of extending an extension primer by using the reverse primer (plus strand) as an extension primer, and the single-stranded oligonucleotide (minus strand) as a template.

While an explanation was made for the case where PCR is executed while using present immobilized oligonucleotide as one of primers and adding the other of primers in amplifying the objective region, when other methods for detecting the objective product (for example, an analytical method capable of comparing the amount of each amplification product obtained by PCR) are executed, PCR may be executed while adding a pair of primers rather than using immobilized oligonucleotide as either one of primers in amplifying the objective region as described above. After conducting such PCR, an amount of the obtained amplification product is determined.

Third step of the present measuring method has repeated steps, and for example, “generated single-stranded DNA (plus strand)” in Step A1 means “generated “free” DNA in a single-stranded state (plus strand)” both in first operation of Third step and in second or later repeated operation of Third step.

In Step B, “generated single-stranded DNA (minus strand)” means “generated “immobilized” DNA in a single-stranded state (plus strand)” both in first operation of Third step and in second or later repeated operation of Third step. However, when Third step further has Step C additionally, it means “generated “immobilized” DNA in a single-stranded state (plus strand)” in first operation of Third step, while it means both “generated “immobilized” DNA in a single-stranded state (plus strand)” and “generated “free” DNA in a single-stranded state (plus strand)” in second or later repeated operation of Third step.

In Step A, “extensionally-formed double-stranded DNA” obtained in each regular step of Third step means “extensionally-formed double-stranded DNA not containing an unmethylated CpG pair in the recognition site of the methylation sensitive restriction enzyme, the site being protected by the masking nucleotide” in the first operation of Third step, while it means both “extensionally-formed double-stranded DNA not containing an unmethylated CpG pair in the recognition site of the methylation sensitive restriction enzyme, the site being protected by the masking nucleotide” and “extensionally-formed double-stranded DNA containing an unmethylated CpG pair in the recognition site of the methylation sensitive restriction enzyme, the site being protected by the masking nucleotide” in the second or later repeated operation of Third step. In Step B, it means “extensionally-formed double-stranded DNA in which a CpG pair is unmethylated in every recognition site of the methylation sensitive restriction enzyme, the site being protected by the masking nucleotide” both in the first operation of Third step and in the second or later repeated operation of Third step.

The same applies to a case where Third step further has Regular step C additionally.

In a case where Third step further has Regular step C additionally, “generated single-stranded DNA (plus strand)” in Step C1 means “generated “free” single-stranded DNA (plus strand)” both in the first operation of Third step and in the second or later repeated operation of Third step.

The present invention also provides a method of measuring methylation rate (that is, the present methylation rate measuring method) further comprising the following two steps as steps of the present measuring method:

(4) Fourth step of amplifying DNA (total amount of methylated DNA and non-methylated DNA) of the objective DNA region to a detectable level by conducting Third step in the present measuring method (including the above modified methods) without conducting Second step of combined step (i) or Second (B) step of combined step (ii) in the present measuring method (including the above modified methods) after conducting First step in the present measuring method (including the above modified methods), and quantifying the amplified DNA; and

(5) Fifth step of calculating a rate of methylated DNA in the objective DNA region based on a difference obtained by comparing the DNA amount quantified by Third step in the present measuring method (including the above modified methods) and the DNA amount quantified in Fourth step.

The methylation rate measuring method may be used in the following situations.

It is known that DNA methylation abnormality occurs in various diseases (for example, cancer), and it is believed that the degree of various diseases can be measured by detecting this DNA methylation abnormality.

For example, when there is a DNA region where methylation occurs at 100% in a genomic DNA contained in a specimen derived from a diseased organism, and the present measuring method or the present methylation rate measuring method is executed for the DNA region, the amount of methylated DNA would increase. For example, when there is a DNA region where methylation does not occur at 100% in a genomic DNA contained in a specimen derived from a diseased organism, and the present measuring method or the present methylation rate measuring method is executed for the DNA region, the amount of methylated DNA would be approximately 0. For example, when there is a DNA region where the methylation rate is low in a genomic DNA contained in a specimen derived from a healthy organism, and a DNA region where the methylation rate is high in a genomic DNA contained in a specimen derived from a diseased organism, and the present measuring method or the present methylation rate measuring method is executed for the DNA region, the amount of methylated DNA would be approximately 0 for a healthy subject, and a significantly higher value than that of a healthy subject would be exhibited by a disease subject, so that the “degree of disease” can be determined based on this difference in value. The “degree of disease” used herein has the same meaning as those commonly used in this field of art, and concretely means, for example, malignancy when the biological specimen is a cell, and means, for example, abundance of disease cells in the tissue when the biological specimen is a tissue. Further, when the biological specimen is plasma or serum, it means the probability that the individual has a disease. Therefore, the present measuring method or the present methylation rate measuring method makes it possible to diagnose various diseases by examining methylation abnormality.

Restriction enzymes, primers or probes that can be used in various methods for measuring a methylated DNA amount in an objective region, and for measuring a methylation rate in the present measuring method or the present methylation rate measuring method are useful as reagents of a detection kit. The present invention also provides a detection kit containing these restriction enzymes, primers or probes as reagents, and a detection chip in which these primers or probes are immobilized on a carrier, and a scope of the present measuring method or the present methylation rate measuring method includes use in the form of the detection kit or the detection chip as described above utilizing the substantial principle of such a method.

### EXAMPLES

In the following, the present invention will be described in detail by way of examples, however, the present invention will not be limited to these examples.

### Example 1

Methylated oligonucleotide GPR7-2079-2176/98 mer-M(7) consisting of the nucleotide sequence of SEQ ID NO:17 in which the recognition site of HpaII is methylated, and unmethylated oligonucleotide GPR7-2079-2176/98 mer-UM consisting of the nucleotide sequence of SEQ ID NO:18 in which the recognition site of HpaII is not methylated were synthesized, and 0.001 pmol/10 μL TE buffer solutions were prepared respectively.

Methylated Oligonucleotide GPR7-2079-2176/98mer-M(7) in which the Recognition Site of HpaII is Methylated, Wherein N Represents Methylated Cytosine:

(SEQ ID NO: 17)

5′-GTTGGCCACTGCGGAGTCGNGCNGGGTGGCNGGCCGCACCTACAGNG
CCGNGNGNGCGGTGAGCCTGGCCGTGTGGGGGATCGTCACACTCGTCGTG
C-3′

Unmethylated Oligonucleotide GPR7-2079-2176/98 mer-UM in which the Recognition Site of HpaII is not Methylated:

(SEQ ID NO: 18)

5′-GTTGGCCACTGCGGAGTCGCGCCGGGTGGCCGGCCGCACCTACAGCG
CCGCGCGCGCGGTGAGCCTGGCCGTGTGGGGGATCGTCACACTCGTCGTG
C-3′

Also, 5′-end biotin-labeled oligonucleotide Bio-GPR7-2176R consisting of the nucleotide sequence of SEQ ID NO:19 was synthesized, and a 0.1 μM TE buffer solution was prepared.

### 5′-End Biotin-Labeled Oligonucleotide Bio-GPR7-2176R:

| | |
|---|---|
| 5′-GCACGACGAGTGTGACGATC-3′ | (SEQ ID NO: 19) |

Each 10 μL of solution of either the obtained methylated oligonucleotide or unmethylated oligonucleotide was added with 1 μL of the above 5′-end biotin-labeled oligonucleotide solution, and 2 μL of an annealing buffer (330 mM Tris-Acetate pH 7.9, 660 mM KOAc, 100 mM MgOAc₂, 5 mM Dithiothreitol), and further the resultant mixture was added with sterilized ultrapure water to make the liquid volume 20 μL, and mixed. For methylated oligonucleotide and unmethylated oligonucleotide, three samples for each were prepared. The obtained mixture was heated at 95° C. for 5 minutes. Thereafter, the mixture was rapidly cooled to 50° C., and kept at that temperature for 5 minutes. Then, after incubation at 37° C. for 5 minutes, the temperature was restored to room temperature.

Next, the previously prepared mixture was added to a PCR tube coated with streptavidin as described above, and kept at 37° C. for 5 minutes.

Then, after removing the solution from the PCR tube, 100 μL of a washing buffer [0.05% Tween20-containing phosphate buffer (1 mM KH₂PO₄, 3 mM Na₂HPO.7H₂O, 154 mM NaCl pH7.4)] was added, and the buffer was removed by pipetting. This operation was repeated another two times.

The following three types of treatments were conducted on the obtained single-stranded DNA selected in the manner as described above.

Group A (no treatment group): The single-stranded DNA prepared as described above was added with 3 μL of a 10× buffer suited for HpaII and HhaI (330 mM Tris-Acetate pH 7.9, 660 mM KOAc, 100 mM MgOAc₂, 5 mM Dithiothreitol) and 3 μL of 10×BSA (Bovine serum albumin 1 mg/ml), and the resultant mixture was added with sterilized ultrapure water to make the liquid volume 30 μL.

Group B (HpaII digestion treatment group): The single-stranded DNA prepared as described above was added with 15 U of HpaII, 3 μL of 10× buffer suited for HpaII and HhaI (330 mM Tris-Acetate pH 7.9, 660 mM KOAc, 100 mM MgOAc₂, 5 mM Dithiothreitol), and 3 μL of 10×BSA (Bovine serum albumin 1 mg/ml), and the resultant mixture was added with sterilized ultrapure water to make the liquid volume 30 μL.

Group C (Masking oligonucleotide addition and HpaII digestion treatment group): The single-stranded DNA prepared as described above was added with 15 U of HpaII, 3 μL of a 10× buffer suited for HpaII and HhaI (330 mM Tris-Acetate pH 7.9, 660 mM KOAc, 100 mM MgOAc2, 5 mM Dithiothreitol), 3 μL of 10×BSA (Bovine serum albumin 1 mg/ml), and 5 pmol of masking oligonucleotide MA having a nucleotide sequence of SEQ ID NO:20, and the resultant mixture was added with sterilized ultrapure water to make the liquid volume 30 μL.

| | |
|---|---|
| Masking oligonucleotide MA: | |
| 5′-GCCACCCGGCGCGA-3′ | (SEQ ID NO:20) |

After incubating each mixture at 37° C. for 16 hours, the supernatant was removed, and 100 μL of a washing buffer [0.05% Tween20-containing phosphate buffer (1 mM KH₂ PO₄, 3 mM Na₂HPO.7H₂O, 154 mM NaCl pH7.4)] was added, and the washing buffer was removed by pipetting. This operation was repeated another two times.

Next, in the above PCR tube, PCR was conducted using a primer consisting of the nucleotide sequence of SEQ ID NO:21 and a primer consisting of the nucleotide sequence of SEQ ID NO:22 (PF2 and PR2) and the following reaction condition, and methylated DNA in an objective DNA region (GPR7-2079-2176, SEQ ID NO:23, also methylated cytosine is represented by C) was amplified.

| | |
|---|---|
| Primer PF2: | |
| 5′-GTTGGCCACTGCGGAGTCG-3′ | (SEQ ID NO: 21) |
| Primer PR2: | |
| 5′-GCACGACGAGTGTGACGATC-3′ | (SEQ ID NO: 22) |

Objective DNA region GPR7-2079-2176:

(SEQ ID NO: 23)

5′-GTTGGCCACTGCGGAGTCGCGCCGGGTGGCCGGCCGCACCTACAGCG
CCGCGCGCGCGGTGAGCCTGGCCGTGTGGGGGATCGTCACACTCGTCGTG
C-3′

As a reaction solution of PCR, a mixture prepared by mixing DNA which is a template with each 3 μL of solutions of the primer consisting of the nucleotide sequence of SEQ ID NO: 21 and the primer consisting of the nucleotide sequence of SEQ ID NO:22 prepared to 3 μM, each 3 μL of 2 mM dNTPs, 3 μL of a buffer (100 mM Tris-HCl pH 8.3, 500 mM KCl, 15 mM MgCl₂, 0.01% Gelatin), 0.15 μL of 5 U/μL thermostable DNA polymerase (AmpliTaq Gold), and 6 μL of a 5N betaine aqueous solution, and adding sterilized ultrapure water to make the liquid volume 30 μL was used. The PCR was conducted in such a condition that the reaction solution was kept at 95° C. for 10 minutes, followed by 20 cycles each including 30 seconds at 95° C., 30 seconds at 59° C. and 45 seconds at 72° C.

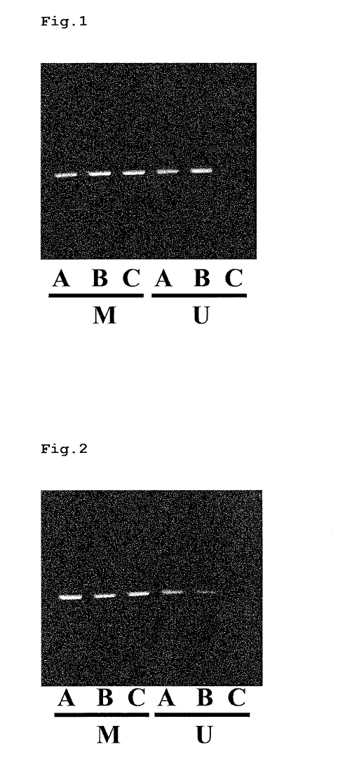

After conducting PCR, DNA amplification was examined by 1.5% agarose gel electrophoresis. The result is shown in FIG. 1. In A treatment group (no treatment group) and B treatment group (HpaII treatment group), the DNA amplification was observed both in methylated oligonucleotide GPR7-2079-2176/98mer-M(7)(M) in which the recognition site of HpaII is methylated and in unmethylated oligonucleotide GPR7-2079-2176/98 mer-UM(U) in which the recognition site of HpaII is not methylated, and amplified products thereof (objective DNA region:GPR7-2079-2176) were obtained. In C treatment group (masking oligonucleotide addition and HpaII digestion treatment group), the DNA amplification was observed and an amplified product thereof (objective DNA region:GPR7-2079-2176) was obtained in the case of methylated oligonucleotide GPR7-2079-2176/98mer-M(7)(M) in which the recognition site of HpaII is methylated, however, the DNA amplification was not observed and an amplified product thereof was not obtained in the case of unmethylated oligonucleotide GPR7-2079-2176/98 mer-UM(U) in which the recognition site of HpaII is not methylated.

From the above description, it was confirmed that single-stranded DNA containing the objective DNA region can be selected, and only methylated DNA can be amplified to a detectable level without amplifying unmethylated DNA in the objective DNA region, and an amount of amplified DNA can be quantified by adding masking oligonucleotide and treating with a methylation sensitive restriction enzyme.

### Example 2

Methylated oligonucleotide consisting of the nucleotide sequence of SEQ ID NO:17 in which the recognition site of HpaII is methylated GPR7-2079-2176/98 mer-M(7), and unmethylated oligonucleotide consisting of the nucleotide sequence of SEQ ID NO:18 in which the recognition site of HpaII is not methylated GPR7-2079-2176/98 mer-UM were synthesized, and 0.001 pmol/10 μL rat serum solutions were prepared respectively.

Methylated Oligonucleotide in which the Recognition Site of HpaII is Methylated GPR7-2079-2176/98mer-M(7), Wherein N Represents Methylated Cytosine:

(SEQ ID NO: 17)

5′-GTTGGCCACTGCGGAGTCGNGCNGGGTGGCNGGCCGCACCTACAGNG
CCGNGNGNGCGGTGAGCCTGGCCGTGTGGGGGATCGTCACACTCGTCGTG
C-3′

Unmethylated Oligonucleotide in which the Recognition Site of HpaII is not Methylated GPR7-2079-2176/98mer-UM:

(SEQ ID NO: 18)

5′-GTTGGCCACTGCGGAGTCGCGCCGGGTGGCCGGCCGCACCTACAGCG
CCGCGCGCGCGGTGAGCCTGGCCGTGTGGGGGATCGTCACACTCGTCGTG
C-3′

Also, 5′-end biotin-labeled oligonucleotide Bio-GPR7-2176R consisting of the nucleotide sequence of SEQ ID NO:19 was synthesized, and a 0.1 μM TE buffer solution was prepared.

5′-end biotin-labeled oligonucleotide Bio-GPR7-2176R:

| | |
|---|---|
| 5′-GCACGACGAGTGTGACGATC-3′ | (SEQ ID NO: 19) |

Each 10 μL of solution of either the obtained methylated oligonucleotide or unmethylated oligonucleotide was added with 1 μL of the above 5′-end biotin-labeled oligonucleotide solution, and 2 μL of an annealing buffer (330 mM Tris-Acetate pH 7.9, 660 mM KOAc, 100 mM MgOAc₂, 5 mM Dithiothreitol), and further the resultant mixture was added with sterilized ultrapure water to make the liquid volume 20 μL, and mixed. For methylated oligonucleotide and unmethylated oligonucleotide, three samples for each were prepared. The obtained mixture was heated at 95° C. for 5 minutes. Thereafter, the mixture was rapidly cooled to 50° C., and kept at that temperature for 5 minutes. Then, after incubation at 37° C. for 5 minutes, the temperature was restored to room temperature.

Next, the previously prepared mixture was added to a PCR tube coated with streptavidin as described above, and kept at 37° C. for 5 minutes.

Then, after removing the solution from the PCR tube, 100 μL of a washing buffer [0.05% Tween20-containing phosphate buffer (1 mM KH₂PO₄, 3 mM Na₂HPO.7H₂O, 154 mM NaCl pH7.4)] was added, and the buffer was removed by pipetting. This operation was repeated another two times.

The following three types of treatments were conducted on the obtained single-stranded DNA selected in the manner as described above.

Group A (no treatment group): The single-stranded DNA prepared as described above was added with 3 μL of a 10× buffer suited for HpaII and HhaI (330 mM Tris-Acetate pH 7.9, 660 mM KOAc, 100 mM MgOAc₂, 5 mM Dithiothreitol) and 3 μL of 10×BSA (Bovine serum albumin 1 mg/ml), and the resultant mixture was added with sterilized ultrapure water to make the liquid volume 30 μL.

Group B (HpaII digestion treatment group): The single-stranded DNA prepared as described above was added with 15 U of HpaII, and 3 μL of 10× buffer suited for HpaII and HhaI (330 mM Tris-Acetate pH 7.9, 660 mM KOAc, 100 mM MgOAc₂, 5 mM Dithiothreitol), and 3 μL of 10×BSA (Bovine serum albumin 1 mg/ml), and the resultant mixture was added with sterilized ultrapure water to make the liquid volume 30 μL.

Group C (Masking oligonucleotide addition and HpaII digestion treatment group): The single-stranded DNA prepared as described above was added with 15 U of HpaII, 3 μL of a 10× buffer suited for HpaII and HhaI (330 mM Tris-Acetate pH 7.9, 660 mM KOAc, 100 mM MgOAc₂, 5 mM Dithiothreitol), 3 μL of 10×BSA (Bovine serum albumin 1 mg/ml), and 5 pmol of masking oligonucleotide MA consisting of the nucleotide sequence of SEQ ID NO:20, the resultant mixture was added with sterilized ultrapure water to make the liquid volume 30 μL.

| | |
|---|---|
| Masking oligonucleotide MA: | |
| 5′-GCCACCCGGCGCGA-3′ | (SEQ ID NO: 20) |

After incubating each mixture at 37° C. for 16 hours, the supernatant was removed, and 100 μL of a washing buffer [0.05% Tween20-containing phosphate buffer (1 mM KH₂PO₄, 3 mM Na₂HPO.7H₂O, 154 mM NaCl pH7.4)] was added, and the washing buffer was removed by pipetting. This operation was repeated another two times.

Next, in the above PCR tube, PCR was conducted using a primer consisting of the nucleotide sequence of SEQ ID NO:21 and a primer consisting of the nucleotide sequence of SEQ ID NO:22 (PF2 and PR2) and the following reaction condition, and methylated DNA in an objective DNA region (GPR7-2079-2176, SEQ ID NO.:23, also methylated cytosine is represented by C) was amplified.

| | |
|---|---|
| Primer PF2: | |
| 5′-GTTGGCCACTGCGGAGTCG-3′ | (SEQ ID NO: 21) |
| Primer PR2: | |
| 5′-GCACGACGAGTGTGACGATC-3′ | (SEQ ID NO: 22) |

Objective DNA region GPR7-2079-2176:

(SEQ ID NO: 23)

5′-GTTGGCCACTGCGGAGTCGCGCCGGGTGGCCGGCCGCACCTACAGCG
CCGCGCGCGCGGTGAGCCTGGCCGTGTGGGGGATCGTCACACTCGTCGTG
C-3′

As a reaction solution of PCR, a mixture prepared by mixing DNA which is a template with each 3 μL of solutions of the primer consisting of the nucleotide sequence of SEQ ID NO: 21 and the primer consisting of the nucleotide sequence of SEQ ID NO:22 prepared to 3 μM, each 3 μL of 2 mM dNTPs, 3 μL of a buffer (100 mM Tris-HCl pH 8.3, 500 mM KCl, 15 mM MgCl₂, 0.01% Gelatin), 0.15 μL of 5 U/μL thermostable DNA polymerase (AmpliTaq Gold), and 6 μL of a 5N betaine aqueous solution, and adding sterilized ultrapure water to make the liquid volume 30 μL was used. The PCR was conducted in such a condition that the reaction solution was kept at 95° C. for 10 minutes, followed by 20 cycles each including 30 seconds at 95° C., 30 seconds at 59° C. and 45 seconds at 72° C.

After conducting PCR, DNA amplification was examined by 1.5% agarose gel electrophoresis. The result is shown in FIG. 2. In A treatment group (no treatment group) and B treatment group (HpaII treatment group), the DNA amplification was observed both in methylated oligonucleotide GPR7-2079-2176/98mer-M(7)(M) in which the recognition site of HpaII is methylated and in unmethylated oligonucleotide GPR7-2079-2176/98 mer-UM(U) in which the recognition site of HpaII is not methylated, and amplified products thereof (objective DNA region:GPR7-2079-2176) were obtained. In C treatment group (masking oligonucleotide addition and HpaII digestion treatment group), the DNA amplification was observed and an amplified product thereof (objective DNA region:GPR7-2079-2176) was obtained in the case of methylated oligonucleotide GPR7-2079-2176/98mer-M(7)(M) in which the recognition site of HpaII is methylated, however, the DNA amplification was not observed and amplified product thereof was not obtained in the case of unmethylated oligonucleotide GPR7-2079-2176/98 mer-UM(U) in which the recognition site of HpaII is not methylated.

From the above description, it was confirmed that even when a serum solution is used as a DNA sample derived from genomic DNA contained in a biological specimen, it is possible to select the single-stranded DNA containing objective DNA region likewise the above Example 1, and only methylated DNA can be amplified to a detectable level without amplifying unmethylated DNA in the objective DNA region, and an amount of amplified DNA can be quantified by adding masking oligonucleotide and treating with a methylation sensitive restriction enzyme.

### Example 3

Breast cancer cell strain MCF-7 derived from a mammal (ATCC NO. HTB-22) purchased from ATCC was cultured to confluent in a special culture for a cell strain described in catalogue of ATCC, to obtain about 1×10⁷ cells. The obtained cells were added with 10-times volume of a SEDTA buffer [10 mM Tris-HCl pH 8.0, 10 mM EDTA pH 8.0, 100 mM NaCl] and homogenized. After adding the obtained mixture with 500 μg/mL of proteinase K(Sigma) and sodium dodecyl sulfate in a concentration of 1% (w/v), the mixture was shaken at 55° C. for about 16 hours. After completion of shaking, the mixture was subjected to extraction with phenol [saturated in 1M Tris-HCl, pH 8.0)]/chloroform. The aqueous layer was collected, added with NaCl in a concentration of 0.5N, subjected to ethanol precipitation, and the generated precipitate was collected. The collected precipitate was dissolved in a TE buffer (10 mM Tris, 1 mM EDTA, pH 8.0), added with RNase A (Sigma) in a concentration of 40 μg/ml, and incubated at 37° C. for 1 hour. The incubated mixture was subjected to phenol/chloroform extraction. The aqueous layer was collected, added with NaCl in a concentration of 0.5N, subjected to ethanol precipitation, and the precipitates (genomic DNA) were collected. By rinsing the collected precipitates with 70% ethanol, genomic DNA was obtained,

By conducting PCR using the obtained genomic DNA as a template, and using the following primers and reaction condition, a DNA fragment (DNA fragment X1, consisting of the nucleotide sequence of SEQ ID NO:25, region corresponding to base Nos. 8-480 in LINE1 sequence shown in Genbank Accession No. M80343 and so on) containing the nucleotide sequence of SEQ ID NO:24 (region corresponding to base Nos. 257-352 in LINE1 sequence shown in Genbank Accession No. M80343 and so on) used as a test sample was amplified.

| | | |
|---|---|---|
| PF1: | 5′-GAGCCAAGATGGCCGAATAGG-3′ | (SEQ ID NO: 26) |
| PR1: | 5′-CTGCTTTGTTTACCTAAGCAAGC-3′ | (SEQ ID NO: 27) |

As a reaction solution of PCR, 2 ng of genomic DNA which is a template, each 0.125 μL of a solution of a primer consisting of the nucleotide sequence of SEQ ID NO: 26 and a solution of a primer consisting of the nucleotide sequence of SEQ ID NO: 27 prepared into 100 pmol/μL, each 2.5 μL of 2 mM of dNTPs, 2.5 μL of a 10× buffer (100 mM Tris-HCl pH 8.3, 500 mM KCl, 15 mM MgCl₂, 0.01% Gelatin) and 0.125 μL of 5 U/μL thermostable DNA polymerase were mixed, and added with sterilized ultrapure water to make the liquid volume 25 μL. The PCR was conducted in such a condition that the reaction solution was kept at 95° C. for 10 minutes, followed by 50 cycles each including 30 seconds at 95° C., 60 seconds at 63° C., and 45 seconds at 72° C.

After conducting the PCR, amplification was examined by 1.5% agarose gel electrophoresis, and an objective DNA fragment (473 bp, DNA fragment X1) was cut out, and purified by using QIAGEN QIAquick Gel Extraction Kit (available from QIAGEN).

A part of the obtained DNA fragment X1 was treated with methylation enzyme SssI (available from NEB) to obtain a DNA fragment in which every 5′-CG-3′ is methylated (hereinafter, denoted by DNA fragment Y1). Also in this case, likewise the above case, amplification was examined by 1.5% agarose gel electrophoresis, and an objective DNA fragment (473 bp, DNA fragment Y1) was cut out, and purified by using QIAGEN QIAquick Gel Extraction Kit (available from QIAGEN).

Using the DNA fragment X1 and DNA fragment Y1, the following mixtures of a methylated fragment and an unmethylated fragment were prepared.

TABLE 1

| | Abundance of DNA | Abundance of DNA | |
|---|---|---|---|
| | fragment X1 | fragment Y1 | |
| | containing objective | containing objective | Methylation |
| DNA fragment | DNA region | DNA region | rate |
| I | 100% | 0% | 0% |
| II | 90% | 10% | 10% |
| III | 75% | 25% | 25% |
| IV | 50% | 50% | 50% |
| V | 0% | 100% | 100% |

The following four kinds of solutions were prepared using respective DNA fragments I to V.

Group A (no treatment group): About 25 ng of a DNA fragment was added with 2 μL of a 10× buffer (330 mM Tris-Acetate pH 7.9, 660 mM KOAc, 100 mM MgOAc₂, 5 mM dithiothreitol) suited for HpaII and HhaI, and 2 μL of 10×BSA (Bovine serum albumin 1 mg/ml), and the resultant mixture was added with sterilized ultrapure water to make the liquid volume 20 μL.

Group B (HpaII treatment group): About 25 ng of a DNA fragment was added with 0.5 U of HpaII, 2 μL of a 10× buffer (330 mM Tris-Acetate pH 7.9, 660 mM KOAc, 100 mM MgOAc₂, 5 mM dithiothreitol) suited for HpaII and HhaI, and 2 μL of 10×BSA (Bovine serum albumin 1 mg/ml), and the mixture was then added with sterilized ultrapure water to make the liquid volume 20 μL.

Group C (HhaI treatment group): About 25 ng of a DNA fragment was added with 0.5 U of HhaI, 2 μL of a 10× buffer (330 mM Tris-Acetate pH 7.9, 660 mM KOAc, 100 mM MgOAc₂, 5 mM dithiothreitol) suited for HpaII and HhaI, and 2 μL of 10×BSA (Bovine serum albumin 1 mg/ml), and the mixture was then added with sterilized ultrapure water to make the liquid volume 20 μL.

Group D (HpaII and HhaI treatment group): About 25 ng of a DNA fragment was added with each 0.5 U of HpaII and HhaI, 2 μL of a 10× buffer (330 mM Tris-Acetate pH 7.9, 660 mM KOAc, 100 mM MgOAc₂, 5 mM dithiothreitol) suited for HpaII and HhaI, and 2 μL of 10×BSA (Bovine serum albumin 1 mg/ml), and the mixture was then added with sterilized ultrapure water to make the liquid volume 20 μL.

After incubating each reaction solution at 37° C. for 2 hours, the solution was ×100 diluted by adding sterilized ultrapure water.

Using 5 μL of each diluted solution (an amount corresponding to 62.5 μg of the DNA fragment) as a template, real time PCR was conducted using the following primers PF2 and PR2 and probe T1 in which the 5′-end is labeled with a reporter fluorescent pigment FAM (6-carboxy-fluorescein) and the 3′-end is labeled with a quencher fluorescent pigment TAMRA (6-carboxy-tetramethyl-rhodamine), in order to determine a DNA amount in the region having the nucleotide sequence of SEQ ID NO: 17.

| | |
|---|---|
| <Primers> | |
| PF2 (forward side): | |
| 5′-CACCTGGAAAATCGGGTCACT-3′ | (SEQ ID NO: 28) |
| PR2 (reverse side): | |
| 5′-CGAGCCAGGTGTGGGATATA-3′ | (SEQ ID NO: 29) |
| <Probe> | |
| T1: | |
| 5′-CGAATATTGCGCTTTTCAGACCGGCTT-3′ | (SEQ ID NO: 30) |

As a reaction solution of PCR, 62.5 pg of the DNA fragment which is a template, each 2.5 μL of a solution of a primer consisting of the nucleotide sequence of SEQ ID NO: 28 and a solution of a primer consisting of the nucleotide sequence of SEQ ID NO: 29 prepared to 3 pmol/μL, 2.5 μL of a probe consisting of the nucleotide sequence of SEQ ID NO: 30 prepared to 2.5 pmol/μL, each 2.5 μL of 2 mM of dNTPs, 2.5 μL of a 10×PCR buffer (100 mM Tris-HCl pH 8.3, 500 mM KCl, 15 mM MgCl₂, 0.01% Gelatin), and 0.125 μL of 5 U/μL of thermostable DNA polymerase (AmpliTaq Gold) were mixed, and added with sterilized ultrapure water to make the liquid volume 25 μL. Real time PCR was conducted using Gene Amp 5700 Sequence Detection System (Applied Biosystems). For amplifying the region (DNA) having a nucleotide sequence of base No. 1 to 94 in the nucleotide sequence represented by SEQ ID NO.: 17, after keeping the reaction solution at 95° C. for 10 minutes, real time PCR was conducted with 15 seconds at 95° C. and 60 seconds at 60° C. being one cycle. From the result of real time PCR, the DNA amount of the region was quantified. Tests were conducted three times for each biological specimen.

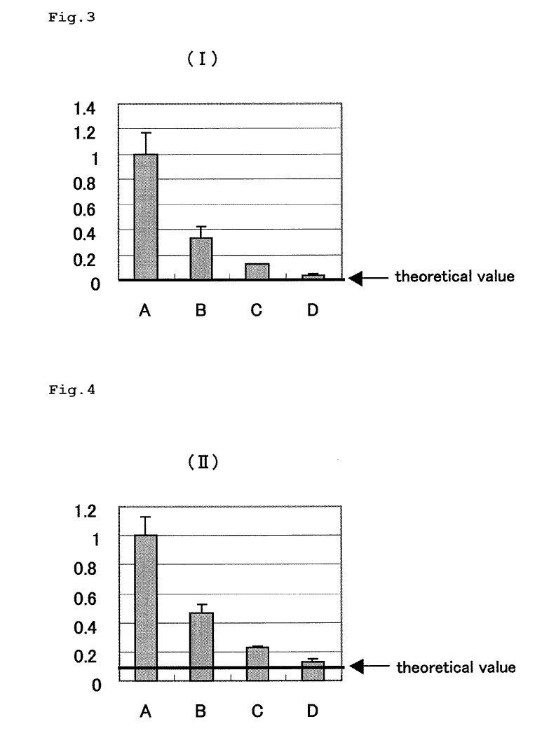

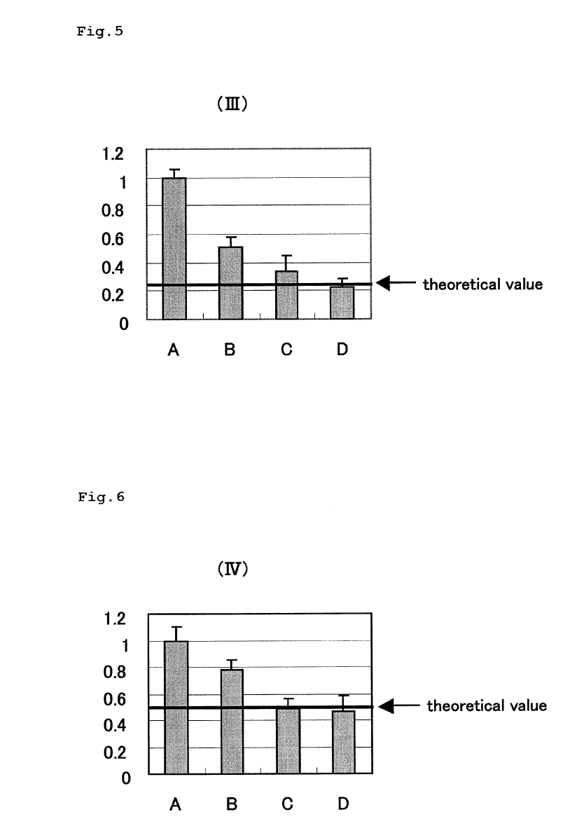

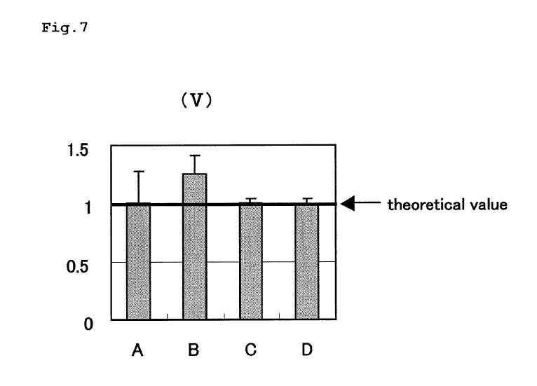

The results are shown in FIGS. 3 to 7. Assuming the DNA amount of the region in Group A as 1, the DNA amounts in the region in other groups are shown. Since FIG. 3 (“I”) is a mixture of a fragment having a methylation rate of 0%, a theoretical value in Group B, Group C and Group D is “0”; since FIG. 4 (“II”) is a fragment having a methylation rate of 10%, a theoretical value in Group B, Group C and Group D is “0.1”; since FIG. 5 (“III”) is a fragment having a methylation rate of 25%, a theoretical value in Group B, Group C and Group D is “0.25”; since FIG. 6 (“IV”) is a fragment having a methylation rate of 50%, a theoretical value in Group B, Group C and Group D is “0.5”; since FIG. 7 (“V”) is a fragment having a methylation rate of 100%, a theoretical value in Group B, Group C and Group D is “1”. As a result of the test, as shown in FIGS. 3 to 7, a value closest to the theoretical value is obtained in Group D, and it was revealed that a digestion treatment with two or more kinds of methylation sensitive enzymes is preferred.

### INDUSTRIAL APPLICABILITY

Based on the present invention, it becomes possible to provide a method of measuring the content of methylated DNA in an objective DNA region in a genomic DNA contained in a biological specimen in a simple and convenient manner, and so on.

### Free Text in Sequence Listing

### SEQ ID NO:17

Designed methylated oligonucleotide for experiment

### SEQ ID NO:18

Designed unmethylated oligonucleotide for experiment

### SEQ ID NO:19

Designed biotinated oligonucleotide for immobilization on support material

### SEQ ID NO:20

Designed oligonucleotide for experiment

### SEQ ID NO:21

Designed oligonucleotide primer for PCR

### SEQ ID NO:22

Designed oligonucleotide primer for PCR

### SEQ ID NO:23

Designed oligonucleotide consist of objective DNA domain (GPR7-2079-2176, methylated cytosin is also shown as C)

### SEQ ID NO:26

Designed oligonucleotide primer for PCR

### SEQ ID NO:27

Designed oligonucleotide primer for PCR

### SEQ ID NO:28

Designed oligonucleotide primer for Real Time PCR

### SEQ ID NO:29

Designed oligonucleotide primer for Real Time PCR

### SEQ ID NO:30

Designed oligonucleotide primer for Real Time PCR

1. A method of measuring the content of methylated DNA in an objective DNA region in a genomic DNA contained in a biological specimen, comprising:
either one of the following combined steps:
(i) combined step (i) comprising:
(1) First step having:
First (A) step of mixing single-stranded DNA (plus strand) containing an objective DNA region with a masking oligonucleotide having a nucleotide sequence complementary to the nucleotide sequence of a recognition site of a methylation sensitive restriction enzyme, thereby generating from a DNA sample derived from genomic DNA contained in a biological specimen single-stranded DNA in which the recognition site of the methylation sensitive restriction enzyme is protected, and
First (B) step of causing base-pairing between the single-stranded DNA (plus strand) containing the objective DNA region protected and generated in First (A) step and a single-stranded immobilized oligonucleotide having a nucleotide sequence complementary to a part (provided that, not containing the objective DNA region) of the 3′-end of the single-stranded DNA, thereby selecting the protected and generated single-stranded DNA, and
(2) Second step of digesting the single-stranded DNA selected in First step with one or more kinds of methylation-sensitive restriction enzyme, and then removing a generated free digest (single-stranded DNA containing one or more unmethylated CpGs in the recognition site of the methylation sensitive restriction enzyme, the site being protected by the masking oligonucleotide);
or,
(ii) combined step (ii) comprising:
(1) First step of causing base-pairing between a single-stranded DNA (plus strand) containing an objective DNA region and a single-stranded immobilized oligonucleotide having a nucleotide sequence complementary to a part (provided that, not containing the objective DNA region) of the 3′-end of the single-stranded DNA, thereby selecting the single-stranded DNA from a DNA sample derived from genomic DNA contained in a biological specimen, and
(2) Second step having:
Second (A) step of mixing the single-stranded DNA selected in First step, with a masking oligonucleotide having a nucleotide sequence complementary to the nucleotide sequence of a recognition site of a methylation-sensitive restriction enzyme, thereby generating a single-stranded DNA in which the recognition site of the methylation-sensitive restriction enzyme is protected, and
Second (B) step of digesting the single-stranded DNA protected and generated in Second (A) step with one or more kinds of methylation-sensitive restriction enzyme, and removing a generated free digest (single-stranded DNA containing one or more unmethylated CpGs in the recognition site of the methylation-sensitive restriction enzyme, the site being protected by the masking oligonucleotide); and
(3) Third step comprising as a pre step of each of the following regular steps:
a step (First pre step) of temporarily separating a single-stranded DNA which is an undigested substance obtained in Second step (single-stranded DNA not containing unmethylated CpG in the recognition site of the methylation sensitive restriction enzyme, the site being protected by the masking oligonucleotide) from both of the single-stranded immobilized oligonucleotide and the masking oligonucleotide, and
a step (Second pre step) having
a step (Second (A) pre step) of causing base-pairing between the generated single-stranded DNA (plus strand) and a single-stranded oligonucleotide, thereby selecting the generated single-stranded DNA and forming DNA in which the selected single-stranded DNA and the single-stranded oligonucleotide are base-paired, and
a step (Second (B) pre step) of making the DNA formed in the step (Second (A) pre step) into double-stranded DNA in which the selected single-stranded DNA has been extended by allowing one extension of a primer by using the selected single-stranded DNA as a template and the single-stranded oligonucleotide as a primer, and
a step (Third pre step) of temporarily separating the double-stranded DNA extensionally-formed in Second pre step (extensionally-formed double-stranded DNA not containing an unmethylated CpG pair in the recognition site of the methylation-sensitive restriction enzyme, the site being protected by the masking oligonucleotide) into a single-stranded DNA (plus strand) and a single-stranded DNA (minus strand), and as regular steps:
(a) Regular step A having Step A1 of selecting the single-stranded DNA by causing base-pairing between the generated single-stranded DNA (plus strand) and the single-stranded immobilized oligonucleotide (minus strand), and Step A2 of extensionally-forming double-stranded DNA from the single-stranded DNA by causing one extension of a primer by using single-stranded DNA selected in Step A1 as a template and the single-stranded immobilized oligonucleotide as the primer, and
(b) Regular step B of extensionally-forming double-stranded DNA from the single-stranded DNA by causing one extension of an extension primer by using the generated single-stranded DNA (minus strand) as a template, and the extension primer (reverse primer) having a nucleotide sequence (plus strand) complementary to a partial nucleotide sequence (minus strand) of nucleotide sequence possessed by the single-stranded DNA (minus strand), wherein the partial nucleotide sequence (minus strand) is positioned on the 3′-end side than the 3′-end of the nucleotide sequence (minus strand) complementary to the nucleotide sequence (plus strand) of the objective DNA region as an extension primer,
wherein the methylated DNA in the objective DNA region is amplified to a detectable level by repeating each regular step after temporarily separating the extensionally-formed double-stranded DNA obtained in each regular step into a single-stranded state, and amount of the amplified DNA is quantified.

2. The method according to claim 1, wherein in First step, base pairing is conducted in a reaction system containing a divalent cation when the single-stranded DNA containing an objective DNA region (plus strand) and the single-stranded immobilized oligonucleotide having a nucleotide sequence complementary to a part (provided that, not containing the objective DNA region) of the 3′-end of the single-stranded DNA are base-paired.

3. The method according to claim 2, wherein the divalent cation is a magnesium ion.

4. The method according to claim 1, further comprising prior to First pre step in Third step:
a step (Additional pre step) of adding into the reaction system a single-stranded oligonucleotide (minus strand) in a free state having a nucleotide sequence complementary to a part of the 3′-end of the single-stranded DNA (plus strand) containing the objective DNA region, and further comprising the following one step as a respective regular step of Third step as described in the item 1:
(c) Regular step C having:
(i) Step C1 of selecting the single-stranded DNA by base-paring the generated single-stranded DNA (plus strand) and the single-stranded oligonucleotide (minus strand) added into the reaction system in Additional pre step, and
(ii) Step C2 of making the single-stranded DNA into extensionally-formed double-stranded DNA by allowing one extension of a primer by using the single-stranded DNA selected in Step C1 as a template and the single-stranded oligonucleotide (minus strand) as a primer.

5. The method according to claim 1, further comprising after First pre step in Third step:
a step (Additional pre step) of adding into the reaction system a single-stranded oligonucleotide (minus strand) in a free state having a nucleotide sequence complementary to a part of the 3′-end of the single-stranded DNA (plus strand) containing the objective DNA region, and further comprising the following one step as a respective regular step of Third step as described in the item 1:
(c) Regular step C having:
(i) Step C1 of selecting the single-stranded DNA by base-paring the generated single-stranded DNA (plus strand) and the single-stranded oligonucleotide (minus strand) added into the reaction system in Additional pre step, and
(ii) Step C2 of making the single-stranded DNA into extensionally-formed double-stranded DNA by allowing one extension of a primer by using the single-stranded DNA selected in Step C1 as a template and the single-stranded oligonucleotide (minus strand) as a primer.

6. The method according to claim 1, further comprising prior to Third pre step in Third step:
a step (Additional pre step) of adding into the reaction system a single-stranded oligonucleotide (minus strand) in a free state having a nucleotide sequence complementary to a part of the 3′-end of the single-stranded DNA (plus strand) containing the objective DNA region, and further comprising the following one step as a respective regular step of Third step as described in the item 1:
(c) Regular step C having:
(i) Step C1 of selecting the single-stranded DNA by base-paring the generated single-stranded DNA (plus strand) and the single-stranded oligonucleotide (minus strand) added into the reaction system in Additional pre step, and
(ii) Step C2 of making the single-stranded DNA into extensionally-formed double-stranded DNA by allowing one extension of a primer by using the single-stranded DNA selected in Step C1 as a template and the single-stranded oligonucleotide (minus strand) as a primer.

7. The method according to claim 1, further comprising after Third pre step in Third step:
a step (Additional pre step) of adding into the reaction system a single-stranded oligonucleotide (minus strand) in a free state having a nucleotide sequence complementary to a part of the 3′-end of the single-stranded DNA (plus strand) containing the objective DNA region, and further comprising the following one step as a respective regular step of Third step as described in the item 1:
(c) Regular step C having:
(i) Step C1 of selecting the single-stranded DNA by base-paring the generated single-stranded DNA (plus strand) and the single-stranded oligonucleotide (minus strand) added into the reaction system in Additional pre step, and
(ii) Step C2 of making the single-stranded DNA into extensionally-formed double-stranded DNA by allowing one extension of a primer by using the single-stranded DNA selected in Step C1 as a template and the single-stranded oligonucleotide (minus strand) as a primer.

8. A method of measuring a methylation rate further comprising the following two steps as steps of the method according claim 1:
(4) Fourth step of amplifying DNA (total amount of methylated DNA and unmethylated DNA) of the objective DNA region to a detectable level by conducting Third step in the method according to any of the items 1 to 7 after conducting First step in the method according to any one of the items 1 to 7 without conducting Second step of combined step (i) or Second (B) step of combined step (ii) in the method according to any one of the items 1 to 7, and quantifying the amplified DNA; and
(5) Fifth step of calculating a rate of methylated DNA in the objective DNA region based on a difference obtained by comparing the DNA amount quantified by Third step according to any one of the items 1 to 7, and the DNA amount quantified in Fourth step.

9. The method according to claim 1, wherein the biological specimen is mammalian serum or plasma.

10. The method according to claim 1, wherein the biological specimen is mammalian blood or bodily secretion.

11. The method according to claim 1, wherein the biological specimen is a cell lysate or a tissue lysate.

12. The method according to claim 1, wherein the DNA sample derived from a genomic DNA contained in a biological specimen is a DNA sample digested in advance with a restriction enzyme whose recognition cleavage site excludes the objective DNA region possessed by the genomic DNA.

13. The method according to claim 1, wherein the DNA sample derived from a genomic DNA contained in a biological specimen is a DNA sample digested with one or more kinds of methylation sensitive restriction enzyme.

14. The method according to claim 1, wherein the DNA sample derived from a genomic DNA contained in a biological specimen is a DNA sample purified in advance.

15. The method according to claim 1, wherein the one or more kinds of methylation sensitive restriction enzyme is a restriction enzyme having its recognition cleavage site in the objective DNA region possessed by a genomic DNA contained in the biological specimen.

16. The method according to claim 1, wherein the one or more kinds of methylation sensitive restriction enzyme is HpaII or HhaI which is a methylation sensitive restriction enzyme.
